(19) United States
(12) Patent Application Publication
(10) Pub. No.: US 2011/0009263 A1
(43) Pub. Date: Jan. 13, 2011

(54) SELECTED SALTS OF 3-(5,6-DIHYDRO-1,4,2-DIOXAZIN-3-YL)-N-[(4,6-DIMETHOXYPYRIMIDIN-2-YL)CARBAMOYL]PYRIDINE-2-SULFONAMIDE, METHOD FOR THE PRODUCTION THEREOF, AND USE THEREOF AS HERBICIDES AND PLANT GROWTH REGULATORS

(75) Inventors: Christian Waldraff, Bad Vilbel (DE); Hansjoerg Kraehmer, Hofheim (DE); Martin Jeffrey Hills, Idstein (DE); Klaus-Helmut Mueller, Duesseldorf (FR); Ulrich Philipp, Odenthal (DE); Ernst Rudolf Gesing, Erkrath (DE)

Correspondence Address: Baker Donelson Bearman, Caldwell & Berkowitz, PC, 920 Massachusetts Ave, NW, Suite 900, Washington, DC, 20001, US

(73) Assignee: Bayer Cropscience AG, Monheim (DE)

(21) Appl. No.: 12/920,547

(22) PCT Filed: Mar. 13, 2009
(86) PCT No.: PCT/EP09/01822

§ 371 Date: Sep. 1, 2010

(30) Foreign Application Priority Data
Mar. 19, 2008 (EP) 08005143.6

(51) Int. Cl. A01N 55/02; C07D 413/14; C07F 3/06; C07F 1/08; A01N 47/36; A01P 13/00

(52) U.S. Cl. 504/126; 544/65; 504/136; 504/191; 504/215; 544/64

(57) ABSTRACT

The present invention relates to selected salts of 3-(5,6-dihydro-1,4,2-dioxazin-3-yl)-N-[(4,6-dimethoxypyrimidin-2-yl)carbamoyl]pyridine-2-sulfonamide, processes for their preparation and to their use as herbicides, in particular as herbicides for the selective control of unwanted harmful plants in leguminous plants, or for the non-selective control of unwanted harmful plants on non-crop land, and also as plant growth regulators, on their own or with safeners and/or in combination with other herbicides, whose use for controlling unwanted harmful plants (such as, for example, broad-leaved/weed grasses) in specific crop plants or as crop protection regulators, with simultaneous and/or sequential application, either as a ready mix or as a tank mix is known.

The present invention relates to selected salts of 3-(5,6-dihydro-1,4,2-dioxazin-3-yl)-N-[(4,6-dimethoxypyrimidin-2-yl)carbamoyl]pyridine-2-sulfonamide, processes for their preparation and to their use as herbicides, in particular as herbicides for the selective control of unwanted harmful plants in leguminous plants, or for non-selective control on non-crop land or in plantation crops, and also as plant growth regulators, on their own or with safeners and/or in combination with other herbicides, whose use for controlling unwanted harmful plants (such as, for example, broad-leaved/weed grasses) in specific crop plants or as crop protection regulators, with simultaneous and/or sequential application, either as a ready mix or as a tank mix is known.

The publication U.S. Pat. No. 5,476,936 discloses certain herbicidally effective N-azinyl-N′-(het)arylsulfonylureas which also include the group of the 3-(5,6-dihydro-1,4,2-dioxazin-3-yl)-N-[pyrimidin-2-yl)carbamoyl]pyridine-2-sulfonamides. U.S. Pat. No. 5,476,936 also describes Na salts of some of the compounds mentioned therein, inter alia Na salts of individual 3-(5,6-dihydro-1,4,2-dioxazin-3-yl)-N-[pyrimidin-2-yl)carbamoyl]pyridine-2-sulfonamides, such as 3-(5,6-dihydro-1,4,2-dioxazin-3-yl)-N-[4,6-dimethoxypyrimidinepyrimidin-2-yl)carbamoyl]pyridine-2-sulfonamide. The effectiveness of these herbicides against harmful plants is at a high level; however, it depends in general on the application rate, the formulation in question, the harmful plants or the spectrum of harmful plants to be controlled in each case, the climatic and soil conditions, etc. A further criterion is the duration of action, or the rate of degradation of the herbicide. Also to be taken into account are, if appropriate, changes in the susceptibility of harmful plants which may occur on prolonged or geographically restricted use of the herbicides. Activity losses in individual plants can only be compensated to a certain extent by higher application rates of the herbicides, for example because this reduces the selectivity of the herbicides, or an improvement in activity is not observed, not even at a higher application rate.

WO 2005/092104 also discloses the non-selective use of some of the herbicidally active N-azinyl-N′-(het)arylsulfonylureas described in U.S. Pat. No. 5,476,963, in particular from the group of the 3-(5,6-dihydro-1,4,2-dioxazin-3-yl)-N-[pyrimidin-2-yl)carbamoyl]pyridine-2-sulfonamides, and also their selective use for controlling weeds in leguminous plants.

Surprisingly, it has now been found that certain selected salts of 3-(5,6-dihydro-1,4,2-dioxazin-3-yl)-N-[(4,6-dimethoxypyrimidin-2-yl)carbamoyl]pyridine-2-sulfonamide are particularly suitable for use as herbicides and/or plant growth regulators. Here, their advantages are not just limited to biological activity, but also extend to improved physicochemical properties, such as, for example solubility, storage stability and miscibility with other herbicides and formulation auxiliaries.

Accordingly, the present invention provides certain agrochemically active salts of 3-(5,6-dihydro-1,4,2-dioxazin-3-yl)-N-[(4,6-dimethoxypyrimidin-2-yl)carbamoyl]pyridine-2-sulfonamide.

Accordingly, the present invention provides compounds of the general formula (I)

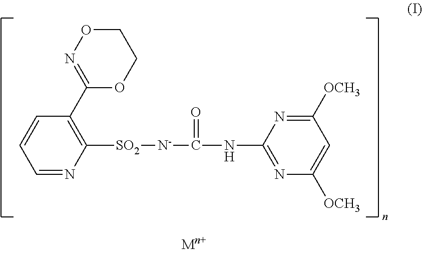

where

the cation M
(a) is a lithium or potassium ion, or
(b) is an alkaline earth metal ion, preferably calcium or magnesium, or
(c) is a transition metal ion, preferably manganese, copper, zinc or iron, or
(d) is an ammonium ion in which optionally one, two, three or all four hydrogen atoms are substituted by identical or different radicals from the group consisting of (C₁-C₄)-alkyl, hydroxy-(C₁-C₄)-alkyl, acylated hydroxy-(C₁-C₄)-alkyl, (C₃-C₆)-cycloalkyl, (C₁-C₄)-alkoxy-(C₁-C₄)-alkyl, hydroxy-(C₁-C₄)-alkoxy-(C₁-C₄)-alkyl, (C₁-C₆)-mercaptoalkyl, phenyl and benzyl, where the radicals mentioned above are optionally substituted by one or more identical or different radicals from the group consisting of halogen, such as F, Cl, Br or I, nitro, cyano, azido, (C₁-C₆)-alkyl, (C₁-C₆)-haloalkyl, (C₃-C₆)-cycloalkyl, (C₁-C₆)-alkoxy, (C₁-C₆)-haloalkoxy and phenyl, and where in each case two substituents at the nitrogen atom together optionally form an unsubstituted or substituted ring, or
(e) is a phosphonium ion, or
(f) is a sulfonium ion, or
(g) is an oxonium ion, or
(h) is a saturated or unsaturated/aromatic nitrogenous heterocyclic ionic compound which has 1-10 carbon atoms in the ring system and is optionally mono- or polycondensed and/or mono- or polysubstituted by (C₁-C₄)-alkyl

and n corresponds to a number 1, 2 or 3.

More preference is given to compounds of the formula (I) in which the cation M
(a) is a lithium or potassium ion, or
(b) is an alkaline earth metal ion, preferably calcium or magnesium, or
(c) is a transition metal ion, preferably manganese, copper, zinc or iron, or
(d) is an ammonium ion in which optionally one, two, three or all four hydrogen atoms are substituted by identical or different radicals from the group consisting of (C₁-C₄)-alkyl, hydroxy-(C₁-C₄)-alkyl, acylated hydroxy-(C₁-C₄)-alkyl, (C₃-C₄)-cycloalkyl, (C₁-C₂)-alkoxy-(C₁-C₂)-alkyl, hydroxy-(C₁-C₂)-alkoxy-(C₁-C₂)-alkyl, (C₁-C₂)-mercaptoalkyl, phenyl and benzyl, where the radicals mentioned above are optionally substituted by one or more identical or different radicals from the group consisting of halogen, such as F, Cl, Br or I, nitro, cyano, azido, (C₁-C₂)-alkyl, (C₁-C₂)-haloalkyl, (C₃-C₄)-cycloalkyl, (C₁-C₂)-alkoxy, (C₁-C₂)-haloalkoxy and phenyl, and where in each case two substituents at the nitrogen atom together optionally form an unsubstituted or substituted ring, or
(e) is a quaternary phosphonium ion, preferably tetra-((C₁-C₄)-alkyl)phosphonium or tetraphenylphosphonium, where the (C₁-C₄)-alkyl radicals and the phenyl radicals are optionally mono- or polysubstituted by identical or different radicals from the group consisting of halogen, such as F, Cl, Br or I, (C₁-C₂)-alkyl, (C₁-C₂)-haloalkyl, (C₃-C₄)-cycloalkyl, (C₁-C₂)-alkoxy and (C₁-C₂)-haloalkoxy, or
(f) is a tertiary sulfonium ion, preferably tri-((C₁-C₄)-alkyl)sulfonium or triphenylsulfonium, where the (C₁-C₄)-alkyl radicals and the phenyl radicals are optionally mono- or polysubstituted by identical or different radicals from the group consisting of halogen, such as F, Cl, Br or I, (C₁-C₂)-haloalkyl, (C₃-C₄)-cycloalkyl, (C₁-C₂)-alkoxy and (C₁-C₂)-haloalkoxy, or
(g) is a tertiary oxonium ion, preferably tri-((C₁-C₄)-alkyl)oxonium, where the (C₁-C₄)-alkyl radicals are optionally mono- or polysubstituted by identical or different radicals from the group consisting of halogen, such as F, Cl, Br or I, (C₁-C₂)-alkyl, (C₁-C₂)-haloalkyl, (C₃-C₄)-cycloalkyl, (C₁-C₂)-alkoxy and (C₁-C₂)-haloalkoxy, or
(h) is a cation from the group of the following heterocyclic compounds, such as, for example, pyridine, quinoline, 2-methylpyridine, 3-methylpyridine, 4-methylpyridine, 2,4-dimethylpyridine, 2,5-dimethylpyridine, 2,6-dimethylpyridine, 5-ethyl-2-methylpyridine, piperidine, pyrrolidine, morpholine, thiomorpholine, pyrrole, imidazole, 1,5-diazabicyclo[4.3.0]non-5-ene (DBN), 1,8-diazabicyclo[5.4.0]undec-7-ene (DBU)

and n corresponds to a number 1, 2 or 3.

Preference is given to compounds of the formula (I) in which the cation M is a potassium ion, a lithium ion, a magnesium ion, a calcium ion, an NH₄⁺ ion, a (2-hydroxyeth-1-yl)ammonium ion, a bis-N,N-(2-hydroxyeth-1-yl)ammonium ion, a tris-N,N,N-(2-hydroxyeth-1-yl)ammonium ion, a methylammonium ion, a dimethylammonium ion, a trimethylammonium ion, a tetramethylammonium ion, an ethylammonium ion, a diethylammonium ion, a triethylammonium ion, a tetraethylammonium ion, an isopropylammonium ion, a diisopropylammonium ion, a tetrapropylammonium ion, a tetrabutylammonium ion, a 2-(2-hydroxyeth-1-oxy)eth-1-ylammonium ion, a di(2-hydroxyeth-1-yl)ammonium ion, a trimethylbenzylammonium ion, a tri-((C₁-C₄)-alkyl)sulfonium ion or a tri-((C₁-C₄)-alkyl)oxonium ion, a benzylammonium ion, a 1-phenylethylammonium ion, a 2-phenylethylammonium ion, a diisopropylethylammonium ion, a pyridinium ion, a piperidinium ion, an imidazolium ion, a morpholinium ion, a 1,8-diazabicyclo[5.4.0]undec-7-enium ion, a diethanolammonium ion, an ethanolammonium ion, a triethanolammonium ion, a cholinium ion, a chlorocholinium ion or an acetylcholinium ion and n corresponds to a number 1 or 2.

More preference is given to compounds of the formula (I) in which the cation M is a potassium ion, a lithium ion, a magnesium ion, a calcium ion or an NH₄⁺ ion. Particular preference is given to compounds of the formula (I) in which the cation M is a potassium ion, a lithium ion or an NH₄⁺ ion and n corresponds to a number 1 or 2.

Particular preference is given to compounds of the formula (I) in which the cation M is a potassium ion or lithium ion and n is 1.

Very particular preference is given to compounds of the formula (I) in which the cation M is a potassium ion and n is 1.

In the formula (I) and in all subsequent formulae, the carbon-containing radicals, such as alkyl, alkoxy, may in each case be straight-chain or branched, for example methyl, ethyl, n- or i-propyl, n-, i-, t- or 2-butyl.

Cycloalkyl is a carbocyclic saturated ring system having preferably 3-6 carbon atoms, for example cyclopropyl, cyclobutyl, cyclopentyl or cyclohexyl.

The invention also provides all stereoisomers embraced by the compounds of the formula (I) according to the invention, and mixtures thereof. The formula (I) embraces all possible stereoisomers defined by their specific spatial form, such as enantiomers, diastereomers, Z and E isomers.

If, for example, one or more alkenyl groups are present, diastereomers (Z and E isomers) may occur. If, for example, one or more asymmetric carbon atoms are present, enantiomers and diastereomers may occur. Stereoisomers may be obtained from the mixtures resulting from the preparation using customary separation methods. It is also possible to prepare stereoisomers selectively by using stereoselective reactions employing optically active starting materials and/or auxiliaries. Accordingly, the invention also relates to all stereoisomers embraced by the general formula (I) but not shown in their specific stereoform, and to their mixtures.

The above examples of radicals or ranges of radicals which are subsumed under the general terms such as “alkyl” do not constitute a complete enumeration. The general terms also embrace the definitions, given later on below, of radical ranges in groups of preferred compounds, especially radical ranges which embrace specific radicals from the tabular examples.

The general or preferred radical definitions given above apply both to the end products of the formula (I) and, correspondingly, to the starting materials or intermediates required in each case for the preparation. These radical definitions can be combined with one another as desired, i.e. including combinations between the given preferred ranges.

The present invention also provides processes for preparing the salts according to the invention, in particular of compounds of the general formula (I), wherein the corresponding free acid (neutral compound) is reacted with an appropriate base to give the general formula (I) in which the group N⁻M⁺ is replaced by NH.

The salts of the formula (I) according to the invention can be prepared in the manner described above by processes known per se, cf. the process steps shown in EP 521500 and WO 2000/044227 (pp. 24 to 27).

For example, the reaction is carried out in the presence of solvents which are inert under the reaction conditions. Suitable solvents are inorganic solvents, such as water, or organic solvents, for example alcohols, such as, for example, methanol or ethanol, halogenated hydrocarbons, such as dichloromethane, ethers, such as tetrahydrofuran or dioxane, ketones, such as acetone or MIBK (methyl isobutyl ketone), amides, such as DMF, nitriles, such as acetonitrile, and sulfoxides, such as DMSO. The reaction temperatures are generally between 0° C. and the boiling point of the solvent. In some cases, the products of the general formula (I) can be isolated directly in virtually quantitative yield and good purity by filtration or, if they are highly soluble, after distillative removal of the solvents.

Collections of compounds of the formula (I) according to the invention which can be synthesized by the reactions mentioned above may also be prepared in a parallel manner, and this may be effected manually or in a semiautomated or fully automated manner. In this case, it is possible, for example, to automate the procedure of the reaction, the work-up or the purification of the products or of the intermediates. In total, this is to be understood as meaning a procedure as is described, for example, by S. H. DeWitt in “Annual Reports in Combinatorial Chemistry and Molecular Diversity: Automated Synthesis”, volume 1, Verlag Escom 1997, pages 69 to 77.

For carrying out microwave-supported syntheses, it is possible to use a microwave apparatus, for example the “Discover” model from CEM GmbH Mikrowellen-Analysentechnik, Carl-Friedrich-Gauβ-Str. 9, 47475 Kamp-Lintfort, Germany.

A number of commercially available apparatuses as offered by, for example, Stem Corporation, Woodrolfe Road, Tollesbury, Essex, England, H+P Labortechnik GmbH, Bruckmannring 28, 85764 Oberschleiβheim, Germany or Radleys, Shirehill, Saffron Walden, Essex, CB 11 3AZ, England, may be used for the parallel procedure of the reaction and work-up. For the parallel purification of compounds of the general formula (I) according to the invention or of intermediates obtained during the preparation, use may be made, inter alia, of chromatography apparatuses, for example those from ISCO, Inc., 4700 Superior Street, Lincoln, Nebr. 68504, USA.

The apparatuses mentioned lead to a modular procedure in which the individual process steps are automated, but manual operations have to be performed between the process steps. This can be avoided by employing semi-integrated or fully integrated automation systems where the automation modules in question are operated by, for example, robots. Such automation systems can be obtained, for example, from Zymark Corporation, Zymark Center, Hopkinton, Mass. 01748, USA.

In addition to the methods described here, the compounds of the general formula (I) according to the invention may be prepared fully or in part by solid-phase-supported methods. For this purpose, individual intermediates or all intermediates of the synthesis or a synthesis adapted to suit the procedure in question are bound to a synthetic resin. Solid-phase-supported synthesis methods are described extensively in the specialist literature, for example Barry A. Bunin in “The Combinatorial Index”, Academic Press, 1998.

The use of solid-phase-supported synthesis methods permits a number of protocols, which are known from the literature and which for their part may be performed manually or in an automated manner, to be carried out. For example, the “teabag method” (Houghten, U.S. Pat. No. 4,631,211; Houghten et al., Proc. Natl. Acad. Sci, 1985, 82, 5131-5135) in which products from IRORI, 11149 North Torrey Pines Road, La Jolla, Calif. 92037, USA, are employed, may be semiautomated. The automation of solid-phase-supported parallel syntheses is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

The preparation according to the processes described herein affords the salts according to the invention, in particular compounds the formula (I), in the form of collections of substances referred to as libraries. The present invention also provides libraries which comprise at least two compounds according to the invention, in particular compounds of the formula (I).

The compounds of the formula (I) according to the invention have excellent herbicidal activity against a broad spectrum of economically important monocotyledonous and dicotyledonous harmful plants. The active compounds also act efficiently on perennial weeds which produce shoots from rhizomes, rootstocks and other perennial organs and which are difficult to control. In this context, it is immaterial whether the substances are applied by the pre-sowing method, the pre-emergence method or the post-emergence method.

If the compounds of the formula (I) according to the invention are applied to the soil surface prior to germination, then the weed seedlings are either prevented completely from emerging, or the weeds grow until they have reached the cotyledon stage but then their growth stops and, eventually, after three to four weeks have elapsed, they die completely.

If the active compounds are applied post-emergence to the green parts of the plants, growth also stops drastically a very short time after the treatment and the weed plants remain at the development stage of the point in time of application, or they die completely after a certain time, so that in this manner competition by the weeds, which is harmful to the crop plants, is eliminated at a very early point in time and in a sustained manner.

Although the compounds of the formula (I) according to the invention have excellent herbicidal activity against monocotyledonous and dicotyledonous weeds, some crop plants of economically important crops, such as, for example, leguminous plants, are not damaged at all, or only to a negligible extent.

The present compounds of the formula (I) according to the invention have excellent selectivity in leguminous plants, an excellent activity against the harmful plants typically occurring in leguminous plants being surprisingly retained even at low dosages of active compound. In particular, harmful plants which occur in crops of leguminous plants and which are frequently difficult to control, such as species of the genera Amaranthus, Echinochloa, Sorghum, Ipomoea, Pharbitis, Solanum, Setaria, Brachiaria, Lolium, Euphorbia and Abutilon are controlled very effectively.

For these reasons, the present compounds of the formula (I) according to the invention are highly suitable for the selective control of unwanted vegetation in leguminous plants, but also for the non-selective control of unwanted vegetation, for example in permanent crops and plantation crops or on roadsides, squares, industrial sites, airports or railway tracks, or in the burn-down application.

In addition, the compounds of the formula (I) according to the invention have outstanding growth-regulating properties in some crop plants. They engage in the plant metabolism in a regulating manner and can thus be employed for the targeted control of plant constituents and for facilitating harvesting, such as, for example, by provoking desiccation and stunted growth. Furthermore, they are also suitable for generally regulating and inhibiting undesirable vegetative growth. Inhibition of vegetative growth plays an important role in many monocotyledonous and dicotyledonous crops because lodging can be reduced hereby, or prevented completely.

By virtue of their herbicidal and plant growth-regulatory properties, the compounds of the formula (I) according to the invention can also be used for controlling harmful plants in crops of known or still to be developed genetically engineered plants. The transgenic plants generally have particularly advantageous properties, for example resistance to certain pesticides, in particular certain herbicides, resistance to plant diseases or causative organisms of plant diseases, such as certain insects or microorganisms, such as fungi, bacteria or viruses. Other particular properties relate, for example, to the quantity, quality, storage-stability, composition and to specific ingredients of the harvested product. Thus, transgenic plants having an increased starch content or a modified quality of the starch or those having a different fatty acid composition of the harvested product are known.

Likewise, by virtue of their herbicidal and plant growth-regulatory properties, the active compounds can also be used for controlling harmful plants in crops of known or still to be developed plants obtained by mutant selection.

The use of the compounds of the formula (I) according to the invention in economically important leguminous transgenic crops or leguminous crops obtained by mutant selection, in particular soybeans, is preferred.

Preferably, the compounds of the formula (I) according to the invention can be used as herbicides in crops of useful plants which are resistant or which have been made resistant by genetic engineering toward the phytotoxic effects of the compounds of the formula (I) according to the invention, or have been obtained by mutant selection. The compounds of the formula (I) according to the invention may likewise preferably be used as herbicides in crops of useful plants which are a crossbreed of plants which have been made resistant by genetic engineering and plants which have been obtained by mutant selection, as described, for example, in WO 2007/024782.

Conventional ways of preparing novel plants which have modified properties compared to known plants comprise, for example, traditional breeding methods and the generation of mutants.

Alternatively, novel plants having modified properties can be generated with the aid of genetic engineering methods (see, for example, EP-A-0221044, EP-A-0131624). For example, there have been described several cases of
genetically engineered changes in crop plants in order to modify the starch synthesized in the plants (for example WO 92/11376, WO 92/14827, WO 91/19806),
transgenic crop plants which are resistant to certain herbicides of the glufosinate type (cf., for example, EP-A-0242236, EP-A-242246) or the glyphosate type (WO 92/00377) or the sulfonylurea type (EP-A-0257993, U.S. Pat. No. 5,013,659),
transgenic crop plants, for example cotton, having the ability to produce Bacillus thuringiensis toxins (Bt toxins) which impart resistance to certain pests to the plants (EP-A-0142924, EP-A-0193259),
transgenic crop plants having a modified fatty acid composition (WO 91/13972).

Numerous molecular biological techniques which allow the preparation of novel transgenic plants having modified properties are known in principle; see, for example, Sambrook et al., 1989, Molecular Cloning, A Laboratory Manual, 2nd ed. Cold Spring Harbor Laboratory Press, Cold Spring Harbor, N.Y.; or Winnacker “Gene and Klone” [Genes and Clones], VCH Weinheim, 2nd edition 1996, or Christou, “Trends in Plant Science” 1 (1996) 423-431).

In order to carry out such genetic engineering manipulations, it is possible to introduce nucleic acid molecules into plasmids which allow a mutagenesis or a change in the sequence to occur by recombination of DNA sequences. Using the abovementioned standard processes it is possible, for example, to exchange bases, to remove partial sequences or to add natural or synthetic sequences. To link the DNA fragments with each other, it is possible to attach adaptors or linkers to the fragments.

Plant cells having a reduced activity of a gene product can be prepared, for example, by expressing at least one appropriate antisense-RNA, a sense-RNA to achieve a cosuppression effect, or by expressing at least one appropriately constructed ribozyme which specifically cleaves transcripts of the abovementioned gene product. To this end, it is possible to employ both DNA molecules which comprise the entire coding sequence of a gene product including any flanking sequences that may be present, and DNA molecules which comprise only parts of the coding sequence, it being necessary for these parts to be long enough to cause an antisense effect in the cells. It is also possible to use DNA sequences which have a high degree of homology to the coding sequences of a gene product but which are not entirely identical. When expressing nucleic acid molecules in plants, the synthesized protein can be localized in any desired compartment of the plant cell. However, to achieve localization in a certain compartment, it is, for example, possible to link the coding region with DNA sequences which ensure localization in a certain compartment. Such sequences are known to a person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated to whole plants using known techniques. The transgenic plants can in principle be plants of any desired plant species, i.e. both monocotyledonous and dicotyledonous plants.

In this manner, it is possible to obtain transgenic plants which have modified properties by overexpression, suppression or inhibition of homologous (=natural) genes or gene sequences or by expression of heterologous (=foreign) genes or gene sequences.

The compounds of the formula (I) according to the invention can preferably be used in transgenic crops or crops obtained by mutant selection or crossbreeds/hybrids thereof which are resistant to herbicides selected from the group consisting of the sulfonylureas, glufosinate-ammonium or glyphosate-isopropylammonium and analogous active compounds.

When using the compounds of the formula (I) according to the invention in transgenic crops or crops obtained by mutant selection or crossbreeds thereof, in addition to the effects against harmful plants which can be observed in other crops, there are frequently effects which are specific for the application in the respective transgenic crops or crops obtained by mutant selection or crossbreeds thereof, for example a modified or specifically broadened spectrum of weeds which can be controlled, modified application rates which can be used for the application, preferably good combinability with the herbicides to which the transgenic crops are resistant, and an effect on the growth and the yield of the transgenic crop plants. The invention therefore also provides for the use of the compounds of the formula (I) according to the invention as herbicides for controlling harmful plants in transgenic crop plants or crop plants obtained by mutant selection or crossbreeds thereof.

The compounds according to the invention can be applied in the customary formulations, for example in the form of wettable powders, emulsifiable concentrates, sprayable solutions, dusts or granules. The invention therefore also provides herbicidal and plant-growth-regulating compositions comprising the compounds of the formula (I) according to the invention.

The compounds of the formula (I) according to the invention can be formulated in various ways depending on the prevailing biological and/or chemico-physical parameters. Examples of suitable formulation options are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing compositions, granules for broadcasting and soil application, granules (GR) in the form of microgranules, spray granules, coating granules and adsorption granules, water-dispersible granules (WG), water-soluble granules (SG), ULV formulations, micro-capsules and waxes.

These individual formulation types are known in principle and are described, for example, in: Winnacker-Küchler, “Chemische Technologie” [Chemical Technology], Volume 7, C. Hanser Verlag Munich, 4th edition 1986; Wade van Valkenburg, “Pesticide Formulations”, Marcel Dekker, N.Y., 1973; K. Martens, “Spray Drying” Handbook, 3rd ed. 1979, G. Goodwin Ltd. London.

The necessary formulation auxiliaries, such as inert materials, surfactants, solvents and other additives, are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”; 2nd ed., J. Wiley & Sons, N.Y.; C. Marsden, “Solvents Guide”; 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesell., Stuttgart 1976; Winnacker-Küchler, “Chemische Technologie” [Chemical Technology], Volume 7, C. Hanser Verlag Munich, 4th edition 1986.

Based on these formulations it is also possible to produce combinations of the compounds of the formula (I) according to the invention with other pesticidally active substances, for example insecticides, acaricides, herbicides and fungicides, and also with safeners, fertilizers and/or growth regulators, for example in the form of a ready mix or tank mix.

Wettable powders are preparations which are uniformly dispersible in water and which contain, in addition to the active compound and as well as a diluent or inert substance, surfactants of ionic and/or nonionic type (wetting agents, dispersants), for example polyethoxylated alkyl phenols, polyethoxylated fatty alcohols, polyethoxylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurinate. To prepare the wettable powders, the herbicidally active compounds are finely ground, for example in customary apparatuses such as hammer mills, fan mills and air-jet mills, and are mixed simultaneously or subsequently with the formulation auxiliaries.

Emulsifiable concentrates are prepared by dissolving the active compound in an organic solvent, for example butanol, cyclohexanone, dimethylformamide, xylene or else relatively high-boiling aromatics or hydrocarbons or mixtures of the organic solvents, with the addition of one or more surfactants of ionic and/or nonionic type (emulsifiers). Examples of emulsifiers which can be used are: calcium alkylarylsulfonates, such as Ca dodecylbenzenesulfonate, or nonionic emulsifiers, such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters or polyoxyethylene sorbitan esters, for example polyoxyethylene sorbitan fatty acid esters.

Dusts are obtained by grinding the active compound with finely divided solid substances, for example talc, natural clays, such as kaolin, bentonite and pyrophyllite, or diatomaceous earth.

Suspension concentrates can be water- or oil-based. They can be prepared, for example, by wet milling using commercially customary bead mills, with or without the addition of surfactants as already mentioned above, for example, in the case of the other formulation types.

Emulsions, for example oil-in-water emulsions (EW), can be prepared for example by means of stirrers, colloid mills and/or static mixers using aqueous organic solvents and, if desired, surfactants as already mentioned above, for example, in the case of the other formulation types.

Granules can be prepared either by spraying the active compound onto adsorptive, granulated inert material or by applying active-compound concentrates to the surface of carriers such as sand, kaolinites or granulated inert material, by means of adhesive binders, for example polyvinyl alcohol, sodium polyacrylate or else mineral oils. Suitable active compounds can also be granulated in the manner which is customary for the preparation of fertilizer granules, if desired as a mixture with fertilizers.

Water-dispersible granules are generally prepared by the customary processes, such as spray-drying, fluidized-bed granulation, disk granulation, mixing using high-speed mixers, and extrusion without solid inert material.

For the preparation of disk, fluidized-bed, extruder and spray granules, see for example processes in “Spray-Drying Handbook” 3rd ed. 1979, G. Goodwin Ltd., London; J. E. Browning, “Agglomeration”, Chemical and Engineering 1967, pages 147 et. seq.; “Perry's Chemical Engineer's Handbook”, 5th ed., McGraw-Hill, New York 1973, pp. 8-57.

For further details on the formulation of crop protection products, see for example G. C. Klingman, “Weed Control as a Science”, John Wiley and Sons., Inc., New York, 1961, pages 81-96 and J. D. Freyer, S. A. Evans, “Weed Control Handbook”, 5th ed., Blackwell Scientific Publications, Oxford, 1968, pages 101-103.

The agrochemical formulations generally contain from 0.1 to 99% by weight, preferably from 0.1 to 95% by weight, particularly preferably from 0.5 to 90% by weight, of the active compounds of the compounds of the formula (I) according to the invention.

In wettable powders the concentration of active compound is, for example, from about 10 to 90% by weight, the remainder to 100% by weight consisting of customary formulation constituents. In emulsifiable concentrates the concentration of active compound can be from about 1 to 90%, preferably from 5 to 80%, by weight. Formulations in the form of dusts contain from 1 to 30% by weight of active compound, preferably most commonly from 5 to 20% by weight of active compound, while sprayable solutions contain from about 0.05 to 80%, preferably from 2 to 50%, by weight of active compound. In the case of water-dispersible granules, the content of active compound depends partly on whether the active compound is in liquid or solid form and on the granulation auxiliaries, fillers, etc. that are used. In water-dispersible granules the content of active compound, for example, is between 1 and 95% by weight, preferably between 10 and 80% by weight.

In addition, said formulations of active compound may comprise the tackifiers, wetting agents, dispersants, emulsifiers, penetrants, preservatives, antifreeze agents, solvents, fillers, carriers, colorants, antifoams, evaporation inhibitors and pH and viscosity regulators which are customary in each case.

If the extender used is water, it is also possible to use, for example, organic solvents as auxiliary solvents. Suitable liquid solvents are essentially: aromatics, such as xylene, toluene, alkylnaphthalenes, chlorinated aromatics or chlorinated aliphatic hydrocarbons, such as chlorobenzenes, chloroethylenes, or methylene chloride, aliphatic hydrocarbons, such as cyclohexane or paraffins, for example mineral oil fractions, mineral and vegetable oils, alcohols, such as butanol or glycol, and ethers and esters thereof, ketones, such as acetone, methyl ethyl ketone, methyl isobutyl ketone or cyclohexanone, strongly polar solvents, such as dimethylformamide or dimethyl sulfoxide, and also water.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as sawdust, coconut shells, corn cobs and tobacco stalks; suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydrolyzates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, and also natural phospholipids, such as cephalins and lecithins, and synthetic phospholipids, can be used in the formulations.

The herbicidal action of the compounds of the formula (I) according to the invention can also be improved, for example, by surfactants, preferably by wetting agents from the group of the fatty alcohol polyglycol ethers. The fatty alcohol polyglycol ethers preferably comprise 10-18 carbon atoms in the fatty alcohol radical and 2-20 ethylene oxide units in the polyglycol ether moiety. The fatty alcohol polyglycol ethers may be present in nonionic form, or ionic form, for example in the form of fatty alcohol polyglycol ether sulfates, which may be used, for example, as alkali metal salts (for example sodium salts and potassium salts) or ammonium salts, or even as alkaline earth metal salts, such as magnesium salts, such as C₁₂/C₁₄-fatty alcohol diglycol ether sulfate sodium (Genapol® LRO, Clariant GmbH); see, for example, EP-A-0476555, EP-A-0048436, EP-A-0336151 or U.S. Pat. No. 4,400,196 and also Proc. EWRS Symp. “Factors Affecting Herbicidal Activity and Selectivity”, 227-232 (1988). Nonionic fatty alcohol polyglycol ethers are, for example, (C₁₀-C₁₈)-, preferably (C₁₀-C₁₄)-fatty alcohol polyglycol ethers (for example isotridecyl alcohol polyglycol ethers) which comprise, for example, 2-20, preferably 3-15, ethylene oxide units, for example those from the Genapol® X-series, such as Genapol® X-030, Genapol® X-060, Genapol® X-080 or Genapol® X-150 (all from Clariant GmbH).

The present invention further comprises the combination of the compounds of the formula (I) according to the invention with the wetting agents mentioned above from the group of the fatty alcohol polyglycol ethers which preferably contain 10-18 carbon atoms in the fatty alcohol radical and 2-20 ethylene oxide units in the polyglycol ether moiety and which may be present in nonionic or ionic form (for example as fatty alcohol polyglycol ether sulfates). Preference is given to C₁₂/C₁₄-fatty alcohol diglycol ether sulfate sodium (Genapol® LRO, Clariant GmbH) and isotridecyl alcohol polyglycol ether having 3-15 ethylene oxide units, for example from the Genapol® X-series, such as Genapol® X-030, Genapol® X-060, Genapol® X-080 and Genapol® X-150 (all from Clariant GmbH). Furthermore, it is known that fatty alcohol polyglycol ethers, such as nonionic or ionic fatty alcohol polyglycol ethers (for example fatty alcohol polyglycol ether sulfates) are also suitable for use as penetrants and activity enhancers for a number of other herbicides, inter alia for herbicides from the group of the imidazolinones (see for example EP-A-0502014).

Furthermore, it is known that fatty alcohol polyglycol ethers, such as nonionic or ionic fatty alcohol polyglycol ethers (for example fatty alcohol polyglycol ether sulfates) are also suitable for use as penetrants and activity enhancers for a number of other herbicides, inter alia for herbicides from the group of the imidazolinones (see for example EP-A-0502014).

The herbicidal action of the herbicide combinations according to the invention can also be enhanced by using vegetable oils. The term vegetable oils is to be understood as meaning oils of oleaginous plant species, such as soybean oil, rapeseed oil, corn oil, sunflower oil, cottonseed oil, linseed oil, coconut oil, palm oil, thistle oil or castor oil, in particular rapeseed oil, and also their transesterification products, for example alkyl esters, such as rapeseed oil methyl ester or rapeseed oil ethyl ester.

The vegetable oils are preferably esters of C₁₀-C₂₂-, preferably C₁₂-C₂₀-, fatty acids. The C₁₀-C₂₂-fatty acid esters are, for example, esters of unsaturated or saturated C₁₀-C₂₂-fatty acids, in particular those having an even number of carbon atoms, for example erucic acid, lauric acid, palmitic acid and in particular C₁₈-fatty acids, such as stearic acid, oleic acid, linoleic acid or linolenic acid.

Examples of C₁₀-C₂₂-fatty acid esters are esters obtained by reacting glycerol or glycol with the C₁₀-C₂₂-fatty acids contained, for example, in oils of oleaginous plant species, or C₁-C₂₀-alkyl-C₁₀-C₂₂-fatty acid esters which can be obtained, for example, by transesterification of the abovementioned glycerol- or glycol-C₁₀-C₂₂-fatty acid esters with C₁-C₂₀-alcohols (for example methanol, ethanol, propanol or butanol). The transesterification can be carried out by known methods as described, for example, in Römpp Chemie Lexikon, 9th edition, Volume 2, page 1343, Thieme Verlag Stuttgart.

Preferred C₁-C₂₀-alkyl-C₁₀-C₂₂-fatty acid esters are methyl esters, ethyl esters, propyl esters, butyl esters, 2-ethylhexyl esters and dodecyl esters. Preferred glycol- and glycerol-C₁₀-C₂₂-fatty acid esters are the uniform or mixed glycol esters and glycerol esters of C₁₀-C₂₂-fatty acids, in particular fatty acids having an even number of carbon atoms, for example erucic acid, lauric acid, palmitic acid and, in particular, C₁₈-fatty acids, such as stearic acid, oleic acid, linoleic acid or linolenic acid.

In the herbicidal compositions according to the invention, the vegetable oils can be present, for example, in the form of commercially available oil-containing formulation additives, in particular those based on rapeseed oil, such as Hasten® (Victorian Chemical Company, Australia, hereinbelow referred to as Hasten, main ingredient: rapeseed oil ethyl ester), Actirob®B (Novance, France, hereinbelow referred to as ActirobB, main ingredient: rapeseed oil methyl ester), Rako-Binol® (Bayer AG, Germany, hereinbelow referred to as Rako-Binol, main ingredient: rapeseed oil), Renol® (Stefes, Germany, hereinbelow referred to as Renol, vegetable oil ingredient: rapeseed oil methyl ester) or Stefes Mero® (Stefes, Germany, hereinbelow referred to as Mero, main ingredient: rapeseed oil methyl ester).

In a further embodiment, the present invention comprises combinations with the vegetable oils mentioned above, such as rapeseed oil, preferably in the form of commercially available oil-containing formulation additives, in particular those based on rapeseed oil, such as Hasten® (Victorian Chemical Company, Australia, hereinbelow referred to as Hasten, main ingredient: rapeseed oil ethyl ester), Actirob®B (Novance, France, hereinbelow referred to as ActirobB, main ingredient: rapeseed oil methyl ester), Rako-Binol® (Bayer AG, Germany, hereinbelow referred to as Rako-Binol, main ingredient: rapeseed oil), Renol® (Stefes, Germany, hereinbelow referred to as Renol, vegetable oil ingredient: rapeseed oil methyl ester) or Stefes Mero® (Stefes, Germany, hereinbelow referred to as Mero, main ingredient: rapeseed oil methyl ester).

It is possible to use colorants, such as inorganic pigments, for example iron oxide, titanium oxide, Prussian Blue, and organic dyes, such as alizarin dyes, azo dyes and metal phthalocyanine dyes, and trace nutrients such as salts of iron, manganese, boron, copper, cobalt, molybdenum and zinc.

The compounds of the formula (I) according to the invention can be employed as such or in the form of their preparations (formulations) combined with other pesticidally active compounds, such as, for example, insecticides, acaricides, nematicides, herbicides, fungicides, safeners, fertilizers and/or growth regulators, for example as finish formulation or as tank mixes.

Suitable as combination partners for the compounds of the formula (I) according to the invention in formulations of mixtures or in tank mixes are, for example, known, preferably herbicidally active compounds whose action is based on the inhibition of, for example, acetolactate synthase, acetyl-coenzyme-A carboxylase, PS I, PS II, HPPDO, phytoene desaturase, protoporphyrinogen oxidase, glutamine synthetase, cellulose biosynthesis, 5-enolpyruvylshikimate 3-phosphate synthetase. Such compounds and also other compounds which can be used, in some cases having an unknown or a different mechanism of action, are described, for example, in Weed Research 26, 441-445 (1986), or in “The Pesticide Manual”, 12th edition 2000, or 13th edition 2003 or 14h edition 2006/2007, or in the corresponding “e-Pesticide Manual”, version 4 (2006), all published by the British Crop Protection Council, (hereinbelow also referred to in short as “PM”), and in the literature cited therein. Lists of “common names” are also available in “The Compendium of Pesticide Common Names” on the Internet. Examples of herbicides known from the literature which may be combined with the compounds of the formula (I) are, for example, the active compounds from Table 1 below (note: the compounds are referred to either by the “common name” according to the International Organization for Standardization (ISO) or by the chemical name, if appropriate together with a customary code number):

acetochlor; acibenzolar-S-methyl; acifluorfen(-sodium); aclonifen; AD-67; AKH 7088, i.e. [[[1-[5-[2-chloro-4-(trifluoromethyl)phenoxy]-2-nitrophenyl]-2-methoxyethylidene]-amino]oxy]acetic acid and methyl [[[1-[5-[2-chloro-4-(trifluoromethyl)phenoxy]-2-nitrophenyl]-2-methoxyethylidene]amino]oxy]acetate; alachlor; alloxydim(-sodium); ametryn; amicarbazone, amidochlor, amidosulfuron; aminocyclopyrachlor (CAS 858956-08-8); aminopyralid; amitrol; ammonium pelargonate; AMS, i.e. ammonium sulfamat; ancimidol; anilofos; asulam; atrazine; aviglycine; azafenidin, azimsulfuron (DPX-A8947); aziprotryn; barban; BAS 516 H, i.e. 5-fluoro-2-phenyl-4H-3,1-benzoxazin-4-one; beflubutamid (UBH-509), benazolin(-ethyl); bencarbazone; benfluralin; benfuresate; bensulfuron(-methyl); bensulide; bentazone; benzfendizone; benzobicyclon, benzofenap; benzofluor; benzoylprop(-ethyl); benzthiazuron; bialaphos; bifenox; bispyribac(-sodium) (KIH-2023); borax; bromacil; bromobutide; bromofenoxim; bromoxynil; bromuron; buminafos; busoxinone; butachlor; butafenacil; butamifos; butenachlor (KH-218); buthidazole; butralin; butroxydim; butylate; cafenstrole (CH-900); caloxydim; carbetamide; carfentrazone(-ethyl); catechin; CDAA, i.e. 2-chloro-N,N-di-2-propenylacetamid; CDEC, i.e. 2-chloroallyl diethyldithiocarbamate; chlormesulon; chlomethoxyfen; chloramben; chlorazifop-butyl; chlorbromuron; chlorbufam; chlorfenac; chlorfenprop; chlorflurecol(-methyl); chlorflurenol(-methyl); chloridazon; chlorimuron(-ethyl); chlormequat(-chloride); chlornitrofen; chlorophthalim (MK-616); chlorotoluron; chloroxuron; chlorpropham; chlorsulfuron; chlorthal-dimethyl; chlorthiamid; chlortoluron; cinidon(-methyl and -ethyl); cinmethylin; cinosulfuron; clefoxydim; clethodim; clodinafop and its ester derivatives (for example clodinafop-propargyl); clofencet; clomazone; clomeprop; cloprop; cloproxydim; clopyralid; clopyrasulfuron(-methyl); cloransulam(-methyl); cumyluron (JC 940); cyanamide; cyanazine; cycloate; cyclosulfamuron (AC 104); cycloxydim; cycluron; cyhalofop and its ester derivatives (for example the butyl ester, DEH-112); cyperquat; cyprazine; cyprazole; daimuron; 2,4-D; 2,4-DB; dalapon; daminozide; dazomet; n-decanol; desmedipham; desmetryn; di-allate; dicamba; dichlobenil; dichlormid; dichlorprop(-P) salts; diclofop and its esters, such as diclofop-methyl; diclofop-P(-methyl); diclosulam; diethatyl(-ethyl); difenoxuron; difenzoquat(metilsulfate); diflufenican; diflufenzopyr(-sodium); dimefuron; dimepiperate; dimethachlor; dimethametryn; dimethazone; dimethenamid (SAN-582H); dimethenamide-P; dimethylarsinic acid; dimethipin; dimetrasulfuron; dimexyflam; dinitramine; dinoseb; dinoterb; diphenamid; dipropetryn; diquat salts; dithiopyr; diuron; DNOC; eglinazine-ethyl; EL 77, i.e. 5-cyano-1-(1,1-dimethylethyl)-N-methyl-1H-pyrazole-4-carboxamide; endothal; epoprodan; EPTC; esprocarb; ethalfluralin; ethametsulfuron-methyl; ethephon; ethidimuron; ethiozin; ethofumesate; ethoxyfen and its esters (for example ethyl ester, HN-252); ethoxysulfuron; etobenzanid (HW 52); F5231, i.e. N-[2-chloro-4-fluoro-5-[4-(3-fluoropropyl)-4,5-dihydro-5-oxo-1H-tetrazol-1-yl]phenyl]ethanesulfonamide; fenchlorazole(-ethyl); fenclorim; fenoprop; fenoxan, fenoxaprop and fenoxaprop-P and also their esters, for example fenoxaprop-P-ethyl and fenoxaprop-ethyl; fenoxydim; fentrazamide; fenuron; ferrous sulfate; flamprop(-methyl or -isopropyl or -isopropyl-L); flamprop-M(-methyl or -isopropyl); flazasulfuron; floazulate (JV-485); florasulam; fluazifop and fluazifop-P and their esters, for example fluazifop-butyl and fluazifop-P-butyl; fluazolate; flucarbazone(-sodium); flucetosulfuron; fluchloralin; flufenacet; flufenpyr(-ethyl); flumetralin; flumetsulam; flumeturon; flumiclorac(-pentyl); flumioxazin (S-482); flumipropyn; fluometuron; fluorochloridone; fluorodifen; fluoroglycofen(-ethyl); flupoxam (KNW-739); flupropacil (UBIC-4243); flupropanoate; flupyrsulfuron(-methyl)(-sodium); flurenol(-butyl); fluridone; flurochloridone; fluroxypyr(-meptyl); flurprimidol; flurtamone; fluthiacet(-methyl) (KIH-9201); fluthiamide; fluxofenim; fomesafen; foramsulfuron; forchlorfenuron; fosamine; furyloxyfen; gibberillic acid; glufosinate(-ammonium); glyphosate(-isopropylammonium); halosafen; halosulfuron(-methyl); haloxyfop and its esters; haloxyfop-P (=R-haloxyfop) and its esters; HC-252; hexazinone; HNPC-C9908, i.e. methyl 2-[[[[[4-methoxy-6-(methylthio)-2-pyrimidinyl]amino]carbonyl]amino]sulfonyl]benzoate; imazamethabenz(-methyl); imazamox; imazapic; imazapyr; imazaquin and salts, such as the ammonium salt; imazethapyr; imazosulfuron; inabenfide; indanofan; iodosulfuron-methyl(-sodium); ioxynil; isocarbamid; isopropalin; isoproturon; isouron; isoxaben; isoxachlortole; isoxaflutole; isoxapyrifop; karbutilate; lactofen; lenacil; linuron; maleic hyrazide (MH); MBTA; MCPA; MCPB; mecoprop(-P); mefenacet; mefluidide; mepiquat(-chloride); mesosulfuron(-methyl); mesotrione; metam; metamifop; metamitron; metazachlor; methabenzthiazuron; metham; methazole; methoxyphenone; methylarsonic acid; methylcyclopropene; methyldymron; methylisothiocyanate; methabenzthiazuron; metobenzuron; metobromuron; (alpha-)metolachlor; metosulam (XRD 511); metoxuron; metribuzin; metsulfuron-methyl; molinate; monalide; monocarbamide dihydrogensulfate; monolinuron; monuron; monosulfuron; MT 128, i.e. 6-chloro-N-(3-chloro-2-propenyl)-5-methyl-N-phenyl-3-pyridazinamine; MT 5950, i.e. N-[3-chloro-4-(1-methylethyl)-phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclofen; nitralin; nitrofen; nitrophenolate mixture; nitrofluorfen; nonanoic acid; norflurazon; orbencarb; orthosulfamuron; oxabetrinil; oryzalin; oxadiargyl (RP-020630); oxadiazon; oxasulfuron; oxaziclomefone; oxyfluorfen; paclobutrazol; paraquat(-dichloride); pebulate; pelargonic acid; pendimethalin; penoxulam; pentachlorophenol; pentanochlor; pentoxazone; perfluidone; pethoxamid; phenisopham; phenmedipham; picloram; picolinafen; pinoxaden; piperophos; piributicarb; pirifenop-butyl; pretilachlor; primisulfuron(-methyl); probenazole; procarbazone-(sodium); procyazine; prodiamine; profluralin; profoxydim; prohexadione(-calcium); prohydrojasmon; proglinazine(-ethyl); prometon; prometryn; propachlor; propanil; propaquizafop and its esters; propazine; propham; propisochlor; propoxycarbazone(-sodium) (MKH-6561); propyzamide; prosulfalin; prosulfocarb; prosulfuron (CGA-152005); prynachlor; pyraclonil; pyraflufen(-ethyl) (ET-751); pyrasulfotole; pyrazolynate; pyrazon; pyrazosulfuron(-ethyl); pyrazoxyfen; pyribambenz-isopropyl (ZJ 0702); pyrimbambenz-propyl (ZJ 0273); pyribenzoxim; pyributicarb; pyridafol; pyridate; pyriftalid; pyriminobac(-methyl) (KIH-6127); pyrimisulfan (KIH-5996); pyrithiobac(-sodium) (KIH-2031); pyroxasulfone (KIH-485); pyroxofop and its esters (for example propargyl ester); pyroxsulam; quinclorac; quinmerac; quinoclamine; quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; sintofen; SN 106279, i.e. 2-[[7-[2-chloro-4-(trifluoromethyl)phenoxy]-2-naphthalenyl]oxy]propanoic acid and methyl 2-[[7-[2-chloro-4-(trifluoromethyl)phenoxy]-2-naphthalenyl]oxy]propanoate; sulcotrione; sulfentrazone (FMC-97285, F-6285); sulfazuron; sulfometuron(-methyl); sulfosate (ICI-A0224); sulfosulfuron; sulfufenacic (CAS 372137-35-4); TCA(-sodium); tebutam (GCP-5544); tebuthiuron; tecnacene; tefuryltrione; tembotrione; tepraloxydim; terbacil; terbucarb; terbuchlor; terbumeton; terbuthylazine; terbutryn; TFH 450, i.e. N,N-diethyl-3-[(2-ethyl-6-methylpheny)sulfonyl]-1H-1,2,4-triazole-1-carboxamide; thenylchlor (NSK-850); thiafluamide, thiazafluron; thiazopyr (Mon-13200); thidiazimin (SN-24085); thidiazuron; thiencarbazone(-methyl); thifensulfuron(-methyl); thiobencarb; Ti 35; tiocarbazil; topramezone; tralkoxydim;

tri-allate; triasulfuron; triaziflam; triazofenamide; tribenuron(-methyl); triclopyr; tridiphane; trietazine;

trifloxysulfuron(-sodium); trifluralin; triflusulfuron and esters (for example methyl ester, DPX-66037); trimeturon; trinexapac; tritosulfuron; tsitodef; uniconazole; vernolate; WL 110547, i.e. 5-phenoxy-1[3-(trifluoromethyl)phenyl]-1H-tetrazole; D-489; ET-751; KIH-218; KIH-485; KIH-509; KPP-300; LS 82-556; NC-324; NC-330; DPX-N8189; SC-0774; DOWCO-535; DK-8910; V-53482; PP-600; MBH-001; TH-547; SYN-523; IDH-100; SYP-249; HOK-201; IR-6396; MTB-951; NC-620.

Preferred combination partners for use in soybean crops are compounds which are selective in soybeans, for example:
(a) herbicides which are selective in soybeans against monocotyledonous and dicotyledonous harmful plants, for example trifluralin, metribuzin, clomazone, pendimethalin, metolachlor, flumetsulam, dimethenamid, linuron, ethalfluralin, flufenacet, norflurazon, vernolate, chlortoluron, chlorotoluron, cloransulam and esters, such as the methyl ester, imazethapyr, imazamox, imazaquin; or
(b) herbicides which are selective in soybeans against dicotyledonous harmful plants, for example sulfentrazone, bentazone, thifensulfuron and its esters, in particular the methyl ester, oxyfluorfen, lactofen, fomesafen, flumiclorac and its esters, such as the pentyl ester, acifluorfen and its sodium salt, 2,4-DB and its esters and salts, flumioxazin, benazolin, 2,4-D and its esters and salts, chlorimuron and its esters and salts, such as chlorimuron-ethyl; or
(c) herbicides which are selective in soybeans against monocotyledonous harmful plants, for example sethoxydim, cycloxydim, clethodim, quizalofop-P and its esters, such as the ethyl or tefuryl ester, fenoxaprop-P and its esters, such as the ethyl ester, fluazifop-P and its esters, such as the butyl ester, haloxyfop and haloxyfop-P and their esters, such as the methyl or the etotyl ester, propaquizafop, alachlor; or
(d) non-selective herbicides which can also be used, for example, for specific purposes in soybeans, for example glufosinate, glyphosate, paraquat (salts), such as paraquat dichloride.

Other preferred combination partners are benozalin, fenoxaprop, lactofen, chlortoluron, flufenacet, metribuzin, benfuresate, fentrazamid, mefenacet, diclofop, ioxynil, bromoxynil, amidosulfuron, flurtamone, diflufenican, ethoxysulfuron, flucarbazone, propoxycarbazone, sulcotrione, mesotrione, isoproturon, iodosulfuron, mesosulfuron, foramsulfuron, anilofos, oxaziclomefone, oxadiargyl, isoxaflutole, linuron.

Suitable mixing partners in cases where the application is in the field of the non-selective control of weeds, are other non-selective herbicides, preferably glufosinate and glyphosate, or their respective salts.

Of particular interest is the selective control of harmful plants in leguminous plants. The compounds of the formula (I) according to the invention have very good to sufficient selectivity in leguminous plants; however, in principle and in particular also in the case of mixtures with other less selective herbicides, phytotoxicities may occur. Of particular interest in this respect are combinations of salts according to the invention, in particular of compounds of the formula (I), which comprise the compounds of the formula (I) according to the invention or combinations thereof with other herbicides or pesticides and safeners. The safeners used in an antidotically effective amount reduce the phytotoxic side-effects of the herbicides/pesticides employed.

The safeners are preferably selected from the group consisting of:

A) compounds of the formula (S-I)

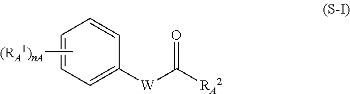

where the symbols and indices have the following meanings:

n_{A} is a natural number from 0 to 5, preferably from 0 to 3;

R_{A}¹ is halogen, (C₁-C₄)-alkyl, (C₁-C₄)-alkoxy, nitro or (C₁-C₄)-haloalkyl;

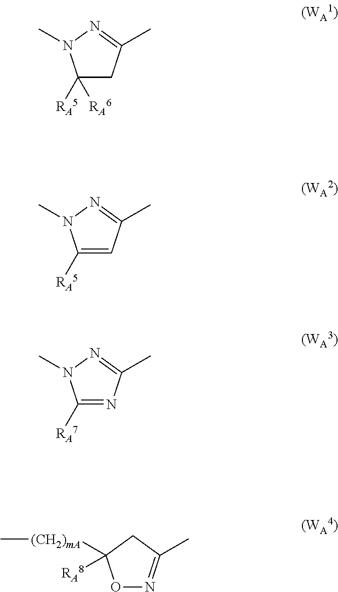

W_{A} is an unsubstituted or substituted divalent heterocyclic radical from the group consisting of partially unsaturated or aromatic five-membered heterocycles having 1 to 3 hetero ring atoms of the type N or O, where at least one nitrogen atom and at most one oxygen atom is present in the ring, preferably a radical from the group consisting of (W_{A}¹) to (W_{A}⁴),

m_{A} is 0 or 1;

R_{A}² is OR_{A}³, SR_{A}³ or NR_{A}³R_{A}⁴ or a saturated

or unsaturated 3- to 7-membered heterocycle having at least one nitrogen atom and up to 3 heteroatoms, preferably from the group consisting of O and S, which is attached via the nitrogen atom to the carbonyl group in (S-I) and which is unsubstituted or substituted by radicals from the group consisting of (C₁-C₄)-alkyl, (C₁-C₄)-alkoxy and optionally substituted phenyl, preferably a radical of the formula OR_{A}³, NHR_{A}⁴ or N(CH₃)₂, in particular of the formula OR_{A}³;

R_{A}³ is hydrogen or an unsubstituted or substituted aliphatic hydrocarbon radical having preferably a total of 1 to 18 carbon atoms;

R_{A}⁴ is hydrogen, (C₁-C₆)-alkyl, (C₁-C₆)-alkoxy or substituted or unsubstituted phenyl;

R_{A}⁵ is H, (C₁-C₈-alkyl, (C₁-C₈-haloalkyl, (C₁-C₄)-alkoxy-(C₁-C₅)-alkyl, cyano or COOR_{A}⁹⁹ where R_{A}⁹ is hydrogen, (C₁-C₈-alkyl, (C₁-C₈-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;

R_{A}⁶, R_{A}⁷, R_{A}⁸ are identical or different and are hydrogen, (C₁-C₈-alkyl, (C₁-C₈-haloalkyl, (C₃-C₁₂)-cycloalkyl or substituted or unsubstituted phenyl;

preferably:

a) compounds of the type of the dichlorophenylpyrazoline-3-carboxylic acid, preferably compounds such as ethyl 1-(2,4-dichlorophenyl)-5-(ethoxycarbonyl)-5-methyl-2-pyrazoline-3-carboxylate (S1-1) (“mefenpyr-diethyl”, see Pestic. Man.), and related compounds, as described in WO 91/07874;

b) derivatives of dichlorophenylpyrazolecarboxylic acid, preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4), ethyl 1-(2,4-dichlorophenyl)-5-phenylpyrazole-3-carboxylate (S1-5) and related compounds, as described in EP-A-333 131 and EP-A-269 806;

c) compounds of the type of the triazolecarboxylic acids, preferably compounds such as fenchlorazole(-ethyl ester), i.e. ethyl 1-(2,4-dichlorophenyl)-5-trichloro-methyl-(1H)-1,2,4-triazole-3-carboxylate (S1-6), and related compounds, as described in EP-A-174 562 and EP-A-346 620;

d) compounds of the type of the 5-benzyl- or 5-phenyl-2-isoxazoline-3-carboxylic acid or the 5,5-diphenyl-2-isoxazoline-3-carboxylic acid, preferably compounds such as ethyl 5-(2,4-dichlorobenzyl)-2-isoxazoline-3-carboxylate (S1-7) or ethyl 5-phenyl-2-isoxazoline-3-carboxylate (S1-8) and related compounds, as described in WO 91/08202, or ethyl 5,5-diphenyl-2-isoxazolinecarboxylate (S1-9) (“isoxadifen-ethyl”) or n-propyl 5,5-diphenyl-2-isoxazolinecarboxylate (S1-10) or ethyl 5-(4-fluorophenyl)-5-phenyl-2-isoxazoline-3-carboxylate (S1-11), as described in the patent application WO-A-95/07897.

B) Quinoline derivatives of the formula (S-II)

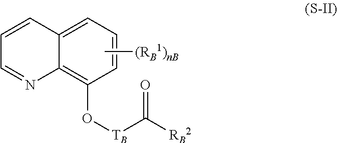

where the symbols and indices have the following meanings:

R_{B}¹ is halogen, (C₁-C₄)-alkyl, (C₁-C₄)-alkoxy, nitro or (C₁-C₄)-haloalkyl;

n_{B} is a natural number from 0 to 5, preferably from 0 to 3;

R_{B}² OR_{B}³, SR_{B}³ or NR_{B}³R_{B}⁴ or a saturated

or unsaturated 3- to 7-membered heterocycle having at least one nitrogen atom and up to 3 heteroatoms, preferably from the group consisting of O and S, which is attached via the nitrogen atom to the carbonyl group in (S-II) and is unsubstituted or substituted by radicals from the group consisting of (C₁-C₄)-alkyl, (C₁-C₄)-alkoxy or optionally substituted phenyl, preferably a radical of the formula OR_{B}³, NHR_{B}⁴ or N(CH₃)₂, in particular of the formula OR_{B}³;

R_{B}³ is hydrogen or an unsubstituted or substituted aliphatic hydrocarbon radical having preferably a total of 1 to 18 carbon atoms;

R_{B}⁴ is hydrogen, (C₁-C₆)-alkyl, (C₁-C₆)-alkoxy or substituted or unsubstituted phenyl;

T_{B} is a (C₁- or C₂)-alkanediyl chain which is unsubstituted or substituted by one or two (C₁-C₄)-alkyl radicals or by [(C₁-C₃)-alkoxy]carbonyl;

preferably:

a) compounds of the type of the 8-quinolinoxyacetic acid (S2), preferably 1-methylhexyl (5-chloro-8-quinolinoxy)acetate (common name “cloquintocet-mexyl” (S2-1) (see Pestic. Man.),

1,3-dimethylbut-1-yl(5-chloro-8-quinolinoxy)acetate (S2-2),

4-allyloxybutyl (5-chloro-8-quinolinoxy)acetate (S2-3),

1-allyloxyprop-2-yl(5-chloro-8-quinolinoxy)acetate-(S2-4),

ethyl (5-chloro-8-quinolinoxy)acetate (S2-5),

methyl (5-chloro-8-quinolinoxy)acetate (S2-6),

allyl (5-chloro-8-quinolinoxy)acetate (S2-7),

2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also their hydrates and salts, as described in WO-A-2002/034048.

b) Compounds of the type of the (5-chloro-8-quinolinoxy)malonic acid, preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.

C) Compounds of the formula (S-III)

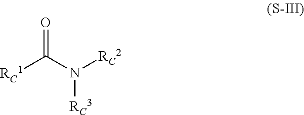

where the symbols and indices have the following meanings:

R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;

R_{C}², R_{C}³ are identical or different and are hydrogen, (C₁-C₄)-alkyl, (C₂-C₄)-alkenyl, (C₂-C₄)-alkynyl, (C₁-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{C}² and R_{C}³ together form a substituted or unsubstituted heterocyclic ring,

preferably an oxazolidine, thiazolidine, piperidine, morpholine, hexahydropyrimidine or benzoxazine ring;

preferably:

active compounds of the type of the dichloroacetamides which are frequently used as pre-emergence safeners (soil-acting safeners), such as, for example, “dichlormid” (see Pestic.Man.) (=N,N-diallyl-2,2-dichloroacetamide),

“R-29148” (=3-dichloroacetyl-2,2,5-trimethyl-1,3-oxazolidine from Stauffer),

“R-28725” (=3-dichloroacetyl-2,2-dimethyl-1,3-oxazolidine from Stauffer),

“benoxacor” (see Pestic. Man.) (=4-dichloroacetyl-3,4-dihydro-3-methyl-2H-1,4-benzoxazine),

“PPG-1292” (=N-allyl-N-[(1,3-dioxolan-2-yl)methyl]dichloroacetamide from PPG Industries),

“DKA-24” (=N-allyl-N-[(allylaminocarbonyl)methyl]dichloroacetamide from Sagro-Chem),

“AD-67” or “MON 4660” (=3-dichloroacetyl-1-oxa-3-aza-spiro[4,5]decane from Nitrokemia or Monsanto),

“T1-35” (=1-dichloroacetylazepane from TRI-Chemical RT)

“diclonon” (dicyclonone) or “BAS145138” or “LAB145138” (=3-dichloroacetyl-2,5,5-trimethyl-1,3-diazabicyclo[4.3.0]nonane from BASF) and

“furilazole” or “MON 13900” (see Pestic. Man.) (=(RS)-3-dichloroacetyl-5-(2-furyl)-2,2-dimethyloxazolidine).

D) N-Acylsulfonamides of the formula (S-IV) and their salts

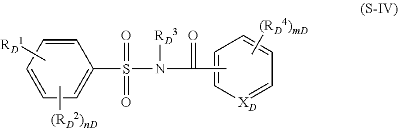

in which

X_{D} is CH or IV;

R_{D}¹ is CO-NR_{D}⁵R_{D}⁶ or NHCO-R_{D}⁷;

R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;

R_{D}³ is hydrogen, (C₁-C₄)-alkyl, (C₂-C₄)-alkenyl or (C₂-C₄)-alkynyl;

R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;

R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group consisting of nitrogen, oxygen and sulfur, where the seven last-mentioned radicals are substituted by v_{D} substituents from the group consisting of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;

R_{D}⁶ is hydrogen, (C₁-C₆)-alkyl, (C₂-C₆)-alkenyl or (C₂-C₆)-alkynyl, where the three last-mentioned radicals are substituted by v_{D} radicals from the group consisting of halogen, hydroxy, (C₁-C₄)-alkyl, (C₁-C₄)-alkoxy and (C₁-C₄)-alkylthio, or

R_{D}⁵ and R_{D}⁶ together with the nitrogen atom carrying them form a pyrrolidinyl or piperidinyl radical;

R_{D}⁷ is hydrogen, (C₁-C₄)-alkylamino, di-(C₁-C₄)-alkylamino, (C₁-C₆)-alkyl, (C₃-C₆)-cycloalkyl, where the 2 last-mentioned radicals are substituted by v_{D} substituents from the group consisting of halogen, (C₁-C₄)-alkoxy, halogen-(C₁-C₆)-alkoxy and (C₁-C₄)-alkylthio and, in the case of cyclic radicals, also (C₁-C₄)-alkyl and (C₁-C₄)-haloalkyl;

n_{D} is 0, 1 or 2;

m_{D} is 1 or 2;

v_{D} is 0, 1, 2 or 3;

from among these, preference is given to compounds of the type of the N-acylsulfonamides, for example of the formula (S-V) below, which are known, for example, from WO 97/45016

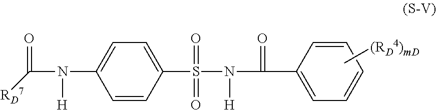

in which

R_{D}⁷ is (C₁-C₆)-alkyl, (C₃-C₆)-cycloalkyl, where the 2 last-mentioned radicals are substituted by v_{D} substituents from the group consisting of halogen, (C₁-C₄)-alkoxy, halogen-(C₁-C₆)-alkoxy and (C₁-C₄)-alkylthio and, in the case of cyclic radicals, also (C₁-C₄)-alkyl and (C₁-C₄)-haloalkyl;

R_{D}⁴ is halogen, (C₁-C₄)-alkyl, (C₁-C₄)-alkoxy, CF₃;

m_{D} is 1 or 2;

v_{D} is 0, 1, 2 or 3;

and also

acylsulfamoylbenzamides, for example of the formula (S-VI) below, which are known, for example, from WO 99/16744,

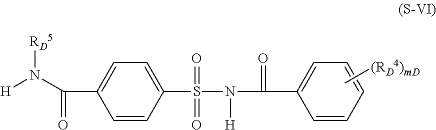

for example those in which

R_{D}⁵=cyclopropyl and (R_{D}⁴)=2-OMe (“cyprosulfamide”, S3-1),

R_{D}⁵=cyclopropyl and (R_{D}⁴)=5-Cl-2-OMe (S3-2),

R_{D}⁵=ethyl and (R_{D}⁴)=2-OMe (S3-3),

R_{D}⁵=isopropyl and (R_{D}⁴)=5-Cl-2-OMe (S3-4) and

R_{D}⁵=isopropyl and (R_{D}⁴)=2-OMe (S3-5);

and also

compounds of the type of the N-acylsulfamoylphenylureas of the formula (S-VII), which are known, for example, from EP-A-365484

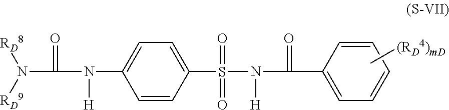

in which

R_{D}⁸ and R_{D}⁹ independently of one another are hydrogen, (C₁-C₈-alkyl, (C₃-C₈)-cycloalkyl, (C₃-C₆)-alkenyl, (C₃-C₆)-alkynyl,

R_{D}⁴ is halogen, (C₁-C₄)-alkyl, (C₁-C₄)-alkoxy, CF₃

m_{D} is 1 or 2;

from among these in particular

1-[4-(N-2-methoxybenzoylsulfamoyl)phenyl]-3-methylurea,

1-[4-(N-2-methoxybenzoylsulfamoyl)phenyl]-3,3-dimethylurea,

1-[4-(N-4,5-dimethylbenzoylsulfamoyl)phenyl]-3-methylurea,

1-[4-(N-naphthoylsulfamoyl)phenyl]-3,3-dimethylurea,

G) active compounds from the class of the hydroxyaromatics and aromatic-aliphatic carboxylic acid derivatives, for example

ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 1,2-dihydro-2-oxo-6-trifluoromethylpyridine-3-carboxamide, 2-hydroxycinnamic acid, 2,4-dichlorocinnamic acid, as described in WO 2004084631, WO 2005015994, WO 2006007981, WO 2005016001;

H) active compounds from the class of the 1,2-dihydroquinoxalin-2-ones, for example

1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydro-quinoxalin-2-one, as described in WO 2005112630,

I) active compounds which, in addition to a herbicidal action against harmful plants, also have safener action on crop plants such as rice, such as, for example, “dimepiperate” or “MY-93” (see Pestic. Man.) (=S-1-methyl-1-phenylethyl piperidine-1-thiocarboxylate), which is known as safener for rice against damage by the herbicide molinate,

“daimuron” or “SK 23” (see Pestic. Man.) (=1-(1-methyl-1-phenylethyl)-3-p-tolyl-urea), which is known as safener for rice against damage by the herbicide imazosulfuron,

“cumyluron”=“JC-940” (=3-(2-chlorophenylmethyl)-1-(1-methyl-1-phenyl-ethyl)urea, see JP-A-60087254), which is known as safener for rice against damage by a number of herbicides,

“methoxyphenone” or “NK 049” (=3,3′-dimethyl-4-methoxybenzophenone), which is known as safener for rice against damage by a number of herbicides,

“CSB” (=1-bromo-4-(chloromethylsulfonyl)benzene) (CAS Reg. No. 54091-06-4 from Kumiai), which is known as safener against damage by a number of herbicides in rice,

K) compounds of the formula (S-IX),
as described in WO-A-1998/38856

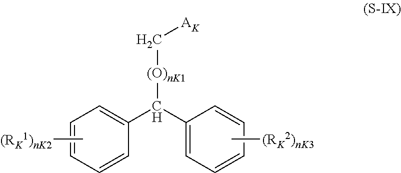

in which the symbols and indices have the following meanings:

R_{K}¹, R_{K}² independently of one another are halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;

A_{K} is COOR_{K}³ or COOR_{K}⁴

R_{K}³, R_{K}⁴ independently of one another are hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl or alkylammonium,

n_{K}¹ is 0 or 1,

n_{K}² , n_{K}³ independently of one another are 0, 1 or 2

preferably: methyl (diphenylmethoxy)acetate (CAS Reg. No.: 41858-19-9),

L) compounds of the formula (S-X),
as described in WO A-98/27049

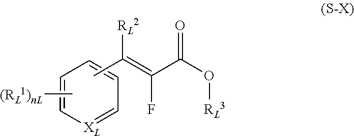

in which the symbols and indices have the following meanings:

X_{L} is CH or N,

n_{L} is, in the case that X=N, an integer from 0 to 4 and,
in the case that X=CH, an integer from 0 to 5,

R_{L}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,

R_{L}² is hydrogen or (C₁-C₄)-alkyl,

R_{L}³ is hydrogen, (C₁-C₈)-alkyl, (C₂-C₄)-alkenyl, (C₂-C₄)-alkynyl or aryl, where each of the carbon-containing radicals mentioned above is unsubstituted or substituted by one or more, preferably by up to three, identical or different radicals from the group consisting of halogen and alkoxy; or salts thereof,

M) active compounds from the class of the 3-(5-tetrazolylcarbonyl)-2-quinolones, for example

1,2-dihydro-4-hydroxy-1-ethyl-3-(5-tetrazolylcarbonyl)-2-quinolone (CAS Reg. No.: 219479-18-2), 1,2-dihydro-4-hydroxy-1-methyl-3-(5-tetrazolylcarbonyl)-2-quinolone (CAS Reg. No.: 95855-00-8), as described in WO-A-1999000020,

N) compounds of the formula (S-XI) or (S-XII),
as described in WO-A-2007023719 and WO-A-2007023764

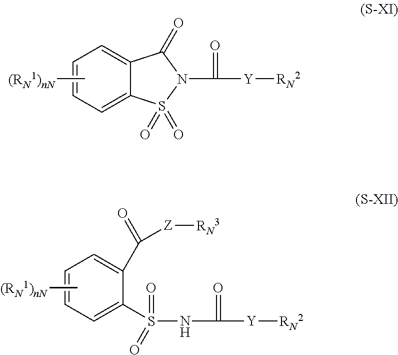

in which

R_{N}¹ is halogen, (C₁-C₄)-alkyl, methoxy, nitro, cyano, CF₃, OCF₃

Y, Z independently of one another are O or S,

n_{N} is an integer from 0 to 4,

R_{N}² is (C₁-C₁₆)-alkyl, (C₂-C₆)-alkenyl, (C₃-C₆)-cycloalkyl, aryl, benzyl, halobenzyl,

R_{N}³ is hydrogen, (C₁-C₆)alkyl,

O) one or more compounds from the group consisting of:

1,8-naphthalic anhydride,

O,O-diethyl S-2-ethylthioethyl phosphorodithioate (disulfoton),

4-chlorophenyl methylcarbamate (mephenate),

O,O-diethyl O-phenyl phosphorothioate (dietholate),

4-carboxy-3,4-dihydro-2H-1-benzopyran-4-acetic acid (CL-304415, CAS Reg. No.: 31541-57-8),

2-propenyl 1-oxa-4-azaspiro[4.5]decane-4-carbodithioate (MG-838, CAS Reg. No.: 133993-74-5),

methyl [(3-oxo-1H-2-benzothiopyran-4(3H)-ylidene)methoxy]acetate (from WO-A-98/13361; CAS Reg. No.: 205121-04-6),

cyanomethoxyimino(phenyl)acetonitrile (cyometrinil),

1,3-dioxolan-2-ylmethoxyimino(phenyl)acetonitrile (oxabetrinil),

4′-chloro-2,2,2-trifluoroacetophenone O-1,3-dioxolan-2-ylmethyloxime (fluxofenim), 4,6-dichloro-2-phenylpyrimidine (fenclorim),

benzyl 2-chloro-4-trifluoromethyl-1,3-thiazole-5-carboxylate (flurazole),

2-dichloromethyl-2-methyl-1,3-dioxolane (MG-191),

including the stereoisomers, and the salts customary in agriculture.

A mixture with other known active compounds, such as fungicides, insecticides, acaricides, nematicides, bird repellents, plant nutrients and soil structure improvers is likewise possible.

Some of the safeners are already known as herbicides and accordingly, in addition to the herbicidal action against harmful plants, also act by protecting the crop plants.

The weight ratios of herbicide (mixture) to safener generally depend on the herbicide application rate and the effectiveness of the safener in question and may vary within wide limits, for example in the range from 200:1 to 1:200, preferably from 100:1 to 1:100, in particular from 20:1 to 1:20. The safeners may be formulated analogously to the compounds of the formula (I) or their mixtures with other herbicides/pesticides and be provided and used as a finished formulation or as a tank mix with the herbicides.

The required application rate of the compound of the formula (I) according to the invention varies depending, inter alia, on external conditions such as temperature, humidity and the type of herbicide used. It can vary within wide limits, for example between 0.001 and 10 000 g/ha or more of active substance; however, it is preferably between 0.5 and 5000 g/ha, particularly preferably between 0.5 and 1000 g/ha and very particularly preferably between 0.5 and 500 g/ha.

The active compounds according to the invention can be used, for example, in connection with the following plants:

dicotyledonous weeds of genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Cucumis, Cucurbita, Daucus, Glycine, Gossypium, Linum, Lycopersicon, Nicotiana, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds according to the invention is in no way restricted to these genera, but also extends in the same manner to other plants.

The salts according to the invention, in particular the compounds of the formula (I), are also suitable for the total control of weeds, for example on industrial terrain and rail tracks, and on paths and areas with or without tree plantings. Similarly, the active compounds according to the invention can be employed for controlling weeds in perennial crops, for example forests, decorative tree plantings, orchards, vineyards, citrus groups, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds of the formula (I) according to the invention have strong herbicidal activity and a broad activity spectrum when used on the soil and on above-ground parts of plants.

The preparation and the use of the active compounds according to the invention is illustrated by the examples below.

### A. SYNTHESIS EXAMPLES

Potassium {[3-(5,6-dihydro-1,4,2-dioxazin-3-yl)pyridin-2-yl]sulfonyl}[(4,6-dimethoxypyrimidin-2-yl)carbamoyl]azanide

10.6 g (0.025 mol) of 3-(5,6-dihydro-1,4,2-dioxazin-3-yl)-N-[(4,6-dimethoxypyrimidin-2-yl)carbamoyl]pyridine-2-sulfonamide are taken up in 750 ml of acetonitrile and heated until a clear solution is formed. With stirring, 2.9 g (0.025 mol) of potassium-tert-butoxide are added. After 16 hours of stirring, a homogeneous suspension has formed, and this suspension is concentrated under reduced pressure. The precipitated solid is filtered off with suction, washed with a little acetonitrile and diethyl ether and dried. This gives 10.0 g of potassium {[3-(5,6-dihydro-1,4,2-dioxazin-3-yl)pyridin-2-yl]sulfonyl}[(4,6-dimethoxypyrimidin-2-yl)carbamoyl]azanide (0.0216 mol, content according to HPLC: 98%, 86.6% of theory) of melting point 162-164° C.

Lithium {[3-(5,6-dihydro-1,4,2-dioxazin-3-yl)pyridin-2-yl]sulfonyl}[(4,6-dimethoxypyrimidin-2-yl)carbamoyl]azanide

10.6 g (0.025 mol) of 3-(5,6-dihydro-1,4,2-dioxazin-3-yl)-N-[(4,6-dimethoxypyrimidin-2-yl)carbamoyl]pyridine-2-sulfonamide are taken up in 750 ml of acetonitrile and heated until a clear solution is formed. With stirring, 2.1 g (0.025 mol) of lithium tert-butoxide are added. After 16 hours of stirring, a homogeneous suspension has formed, and this suspension is concentrated under reduced pressure. The precipitated solid is filtered off with suction, washed with a little acetonitrile and diethyl ether and dried. This gives 11.3 g of lithium {[3-(5,6-dihydro-1,4,2-dioxazin-3-yl)pyridin-2-yl]sulfonyl}[(4,6-dimethoxypyrimidin-2-yl)carbamoyl]azanide (0.0216 mol, content according to HPLC: 96.4%, 100.0% of theory) of melting point 194-196° C.

2-Hydroxy-N-(2-hydroxyethyl)ethanaminium {[3-(5,6-dihydro-1,4,2-dioxazin-3-yl)pyridin-2-yl]sulfonyl}[(4,6-dimethoxypyrimidin-2-yl)carbamoyl]azanide

5.8 g (0.0137 mol) of 3-(5,6-dihydro-1,4,2-dioxazin-3-yl)-N-[(4,6-dimethoxypyrimidin-2-yl)carbamoyl]pyridine-2-sulfonamide are taken up in 300 ml of acetonitrile and heated until a clear solution is formed. With stirring, 1.3 g (0.0124 mol) of diethanolamin are added dropwise, and a white solid soon precipitates out. The mixture is stirred at room temperature for 16 hours. The solid is filtered off with suction, washed with acetonitrile and dried. This gives 6.2 g of 2-hydroxy-N-(2-hydroxyethyl)ethanaminium {[3-(5,6-dihydro-1,4,2-dioxazin-3-yl)pyridin-2-yl]sulfonyl}[(4,6-dimethoxypyrimidin-2-yl)carbamoyl]azanide (0.0116 mol, content according to HPLC: 98.9%, 93.6% of theory).

¹H-NMR (400 MHz, [D6]-DMSO): δ=2.96 (t, 4H); 3.62 (br. t, 4H); 3.80 (s, 6H); 4.16 (m, 2H); 4.40 (m, 2H); 5.09 (br. s, 2H); 5.68 (s, 1H); 7.52 (m, 1H); 7.87 (m, 1H); 8.64 (m, 1H); 8.1-8.9 (br., 1H) ppm

Propan-2-aminium {[3-(5,6-dihydro-1,4,2-dioxazin-3-yl)pyridin-2-yl]sulfonyl}[(4,6-dimethoxypyrimidin-2-yl)carbamoyl]azanide

5.8 g (0.0137 mol) of 3-(5,6-dihydro-1,4,2-dioxazin-3-yl)-N-[(4,6-dimethoxypyrimidin-2-yl)carbamoyl]pyridine-2-sulfonamide are taken up in 300 ml of acetonitrile and heated until a clear solution is formed. With stirring, 800 mg (13.5 mmol) of isopropylamine are added dropwise, and a white solid soon precipitates out. The mixture is stirred at room temperature for 16 hours. The solid is filtered off with suction, washed with acetonitrile and dried. This gives 5.2 g of propan-2-aminium {[3-(5,6-dihydro-1,4,2-dioxazin-3-yl)pyridin-2-yl]sulfonyl}[(4,6-dimethoxypyrimidin-2-yl)carbamoyl]azanide (0.0105 mol, content according to HPLC: 98.0%, 77.9% of theory).

¹H-NMR (400 MHz, [D6]-DMSO): δ=1.15 (d, 6H); 3.28 (m, 1H); 3.79 (s, 6H); 4.16 (m, 2H); 4.39 (m, 2H); 5.65 (s, 1H); 7.48 (m, 1H); 7.5-7.8 (br., 1H); 7.83 (m, 1H); 8.61 (m, 1H) ppm

Ammonium {[3-(5,6-dihydro-1,4,2-dioxazin-3-yl)pyridin-2-yl]sulfonyl}[(4,6-dimethoxypyrimidin-2-yl)carbamoyl]azanide

10.6 g (0.025 mol) of 3-(5,6-dihydro-1,4,2-dioxazin-3-yl)-N-[(4,6-dimethoxypyrimidin-2-yl)carbamoyl]pyridine-2-sulfonamide are taken up in 750 ml of acetonitrile and heated until a clear solution is formed. With stirring, 1.7 g of a 26%-strength aqueous ammonia solution (0.026 mol) are added dropwise, and a white solid soon precipitates out. The mixture is stirred at room temperature for 16 hours and then carefully concentrated under reduced pressure. The white solid is filtered off with suction, washed with acetonitrile and dried. This gives 9.1 g of ammonium {[3-(5,6-dihydro-1,4,2-dioxazin-3-yl)pyridin-2-yl]sulfonyl}[(4,6-dimethoxypyrimidin-2-yl)carbamoyl]azanide (0.0206 mol, content according to HPLC: 100%), 82.5% of theory).

¹H-NMR (400 MHz, [D6]-DMSO): δ=3.84 (s, 6H); 4.18 (m, 2H); 4.43 (m, 2H); 5.83 (br. s, 1H); 6.5-9.5 (br., 4H); 7.64 (br., 1H); 8.01 (br., 1H); 8.70 (m, 1H) ppm

2-Hydroxy-N,N,N-trimethylethanaminium {[3-(5,6-dihydro-1,4,2-dioxazin-3-yl)pyridin-2-yl]sulfonyI}[(4,6-dimethoxypyrimidin-2-yl)carbamoyl]azanide

9.0 g (0.020 mol) of sodium {[3-(5,6-dihydro-1,4,2-dioxazin-3-yl)pyridin-2-yl]sulfonyl}[(4,6-dimethoxypyrimidin-2-yl)carbamoyl]azanide are suspended in 600 ml of methanol. With stirring, 2.8 g (0.020 mol) of cholinium chloride and 100 ml of water are added. The mixture is then heated until a clear solution is formed. The mixture is stirred at room temperature for 16 hours, during which time the solution becomes turbid. The mixture is concentrated to dryness under reduced pressure and taken up in isopropanol. The white solid is filtered off with suction, washed with diethyl ether and dried. This gives 11.1 g of 2-hydroxy-N,N,N-trimethylethanaminium {[3-(5,6-dihydro-1,4,2-dioxazin-3-yl)pyridin-2-yl]sulfonyl}[(4,6-dimethoxypyrimidin-2-yl)carbamoyl]azanide (0.0187 mol, content according to HPLC: 99.2%, 93.3% of theory) as a mixture with sodium chloride.

M.p.: 126-130° C. (decomp.)

2-Chloro-N,N,N-trimethylethanaminium {[3-(5,6-dihydro-1,4,2-dioxazin-3-yl)pyridin-2-yl]sulfonyl}[(4,6-dimethoxypyrimidin-2-yl)carbamoyl]azanide

9.0 g (0.020 mol) of sodium {[3-(5,6-dihydro-1,4,2-dioxazin-3-yl)pyridin-2-yl]sulfonyl}[(4,6-dimethoxypyrimidin-2-yl)carbamoyl]azanide are suspended in 600 ml of methanol. With stirring, 3.2 g (0.020 mol) of 2-chloro-N,N,N-trimethylethanaminium chloride and 100 ml of water are added. The mixture is then heated until a clear solution is formed. The mixture is stirred at room temperature for 16 hours, during which time a precipitate is formed. The mixture is concentrated to dryness under reduced pressure and taken up in isopropanol. The white solid is filtered off with suction, washed with diethyl ether and dried. This gives 9.5 g of 2-chloro-N,N,N-trimethylethanaminium {[3-(5,6-dihydro-1,4,2-dioxazin-3-yl)pyridin-2-yl]sulfonyl}[(4,6-dimethoxypyrimidin-2-yl)carbamoyl]azanide (0.0153 mol, content according to HPLC: 98.5%, 76.7% of theory) as a mixture with sodium chloride.

M.p.: 120-126° C. (decomp.)

2-(Acetyloxy)-N,N,N-trimethylethanaminium {[3-(5,6-dihydro-1,4,2-dioxazin-3-yl)pyridin-2-yl]sulfonyl}[(4,6-dimethoxypyrimidin-2-yl)carbamoyl]azanide

9.0 g (0.020 mol) of sodium {[3-(5,6-dihydro-1,4,2-dioxazin-3-yl)pyridin-2-yl]sulfonyl}[(4,6-dimethoxypyrimidin-2-yl)carbamoyl]azanide are suspended in 600 ml of methanol. With stirring, 3.7 g of (0.020 mol) 2-(acetyloxy)-N,N,N-trimethylethanaminium chloride and 100 ml of water are added. The mixture is then heated until a clear solution is formed. The mixture is stirred at room temperature for 16 hours, during which time the solution becomes turbid. The mixture is concentrated to dryness under reduced pressure and taken up in isopropanol. The white solid is filtered off with suction, washed with diethyl ether and dried. This gives 9.2 g of 2-(acetyloxy)-N,N,N-trimethylethanaminium {[3-(5,6-dihydro-1,4,2-dioxazin-3-yl)pyridin-2-yl]sulfonyl}[(4,6-dimethoxypyrimidin-2-yl)carbamoyl]azanide (0.0131 mol, content according to HPLC: 90.2%, 65.3% of theory)) as a mixture with sodium chloride.

M.p.: >138° C. (decomp.)

2-Hydroxy-N,N-bis(2-hydroxyethyl)ethanaminium {[3-(5,6-dihydro-1,4,2-dioxazin-3-yl)pyridin-2-yl]sulfonyl}[(4,6-dimethoxypyrimidin-2-yl)carbamoyl]azanide

10.0 g (0.0217 mol) of 3-(5,6-dihydro-1,4,2-dioxazin-3-yl)-N-[(4,6-dimethoxypyrimidin-2-yl)carbamoyl]pyridin-2-sulfonamide are taken up in 600 ml of acetonitrile and heated until a clear solution is formed. With stirring, 3.23 g (0.0217 mol) of tris-(2-hydroxyethyl)amine are added dropwise. The mixture is stirred at room temperature for 16 hours, and over time a white solid precipitates out. The solid is filtered off with suction, washed with acetonitrile and dried. This gives 9.2 g of 2-hydroxy-N,N-bis(2-hydroxyethyl)ethanaminium {[3-(5,6-dihydro-1,4,2-dioxazin-3-yl)pyridin-2-yl]sulfonyl}[(4,6-dimethoxypyrimidin-2-yl)carbamoyl]azanide (0.0160 mol, content according to HPLC: 100%, 74.0% of theory).

¹H-NMR (400 MHz, [D6]-DMSO): δ=2.7-3.2 (br. s, 6H); 3.5-3.7 (br. s, 6H); 3.84 (br. s, 6H); 4.18 (m, 2H); 4.43 (m, 2H); 4.5-5.2 (br. 3H); 5.84 (br. s, 1H); 7.65 (br. s, 1H); 8.0 (br. s, 1H); 8.7 (br. m, 1H) ppm

2-Hydroxyethanaminium {[3-(5,6-dihydro-1,4,2-dioxazin-3-yl)pyridin-2-yl]sulfonyl}[(4,6-dimethoxypyrimidin-2-yl)carbamoyl]azanide

5.0 g (10.84 mmol) of 3-(5,6-dihydro-1,4,2-dioxazin-3-yl)-N-[(4,6-dimethoxypyrimidin-2-yl)carbamoyl]pyridin-2-sulfonamide are taken up in 300 ml of acetonitrile and heated until a clear solution is formed. With stirring, 0.66 g (10.84 mmol) of 2-aminoethanol are added dropwise. The mixture is stirred at room temperature for 16 hours, and over time a white solid precipitates out. The solid is filtered off with suction, washed with acetonitrile and dried. This gives 4.2 g of 2-hydroxyethan-aminium {[3-(5,6-dihydro-1,4,2-dioxazin-3-yl)pyridin-2-yl]sulfonyl}[(4,6-dimethoxypyrimidin-2-yl)carbamoyl]azanide (8.3 mmol, content according to HPLC: 95.5%, 76.2% of theory).

¹H-NMR (400 MHz, [D6]-DMSO): δ=2.82 (t, 2H); 3.54 (br. t, 2H); 3.78 (s, 6H); 4.16 (m, 2H); 4.39 (m, 2H); 5.05 (br. s, 1H); 5.64 (s, 1H); 7.1-7.5 (br., 3H); 7.48 (m, 1H); 7.83 (m, 1H); 8.61 (m, 1H) ppm

The compounds described in Table 1 below are obtained as described directly above, or analogously to the above examples.

TABLE 1
Compounds of the general formula (I),
where M denotes the cation of the salt in question
(I)

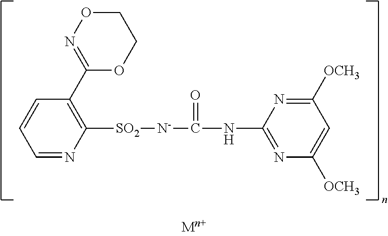

| Compound | M | n |
|---|---|---|
| I-1 | lithium | 1 |
| I-2 | potassium | 1 |
| I-3 | magnesium | 2 |
| I-4 | calcium | 2 |
| I-5 | ammonium | 1 |
| I-6 | methylammonium | 1 |
| I-7 | dimethylammonium | 1 |
| I-8 | tetramethylammonium | 1 |
| I-9 | ethylammonium | 1 |
| I-10 | diethylammonium | 1 |
| I-11 | tetraethylammonium | 1 |
| I-12 | propylammonium | 1 |
| I-13 | tetrapropylammonium | 1 |
| I-14 | isopropylammonium | 1 |
| I-15 | diisopropylammonium | 1 |
| I-16 | butylammonium | 1 |
| I-17 | tetrabutylammonium | 1 |
| I-18 | (2-hydroxyeth-1-yl)ammonium | 1 |
| I-19 | bis-N,N-(2-hydroxyeth-1-yl)ammonium | 1 |
| I-20 | tris-N,N,N-(2-hydroxyeth-1-yl)ammonium | 1 |
| I-21 | 1-phenylethylammonium | 1 |
| I-22 | 2-phenylethylammonium | 1 |
| I-23 | trimethylsulfonium | 1 |
| I-24 | trimethyloxonium | 1 |
| I-25 | pyridinium | 1 |
| I-26 | 2-methylpyridinium | 1 |
| I-27 | 4-methylpyridinium | 1 |
| I-28 | 2,4-dimethylpyridinium | 1 |
| I-29 | 2,6-dimethylpyridinium | 1 |
| I-30 | piperidinium | 1 |
| I-31 | imidazolium | 1 |
| I-32 | morpholinium | 1 |
| I-33 | 1,5-diazabicyclo[4.3.0]non-7-enium | 1 |
| I-34 | 1,8-diazabicyclo[5.4.0]undec-7-enium | 1 |
| I-35 | diethanolammonium | 1 |
| I-36 | ethanolammonium | 1 |
| I-37 | triethanolammonium | 1 |
| I-38 | cholinium | 1 |
| I-39 | chlorocholinium | 1 |
| I-40 | acetylcholinium | 1 |

### B. FORMULATION EXAMPLES

a) A dust is obtained by mixing 10 parts by weight of a compound of the formula (I) according to the invention and 90 parts by weight of talc as inert substance and comminuting the mixture in a hammer mill.

b) A wettable powder which is readily dispersible in water is obtained by mixing 25 parts by weight of a compound of the formula (I) according to the invention, 64 parts by weight of kaolin-containing quartz as inert material, 10 parts by weight of potassium lignosulfonate and 1 part by weight of sodium oleoylmethyltauride as wetting agent and dispersant, and grinding the mixture in a pinned-disk mill.

c) A dispersion concentrate which is readily dispersible in water is obtained by mixing 20 parts by weight of a compound of the formula (I) according to the invention with 6 parts by weight of alkylphenol polyglycol ether (®Triton X 207), 3 parts by weight of isotridecanol polyglycol ether (8 EO) and 71 parts by weight of paraffinic mineral oil (boiling range for example approx. 255 to above 277° C.), and grinding the mixture in a ball mill to a fineness of below 5 micron.

d) An emulsifiable concentrate is obtained from 15 parts by weight of a compound of the formula (I) according to the invention, 75 parts by weight of cyclohexanone as solvent and 10 parts by weight of oxethylated nonylphenol as emulsifier.

e) Water-dispersible granules are obtained by mixing
75 parts by weight of a compound of the formula (I) according to the invention,
10 parts by weight of calcium lignosulfonate,
5 parts by weight of sodium lauryl sulfate,
3 parts by weight of polyvinyl alcohol and
7 parts by weight of kaolin,
grinding the mixture in a pinned-disk mill and granulating the powder in a fluidized bed by spraying on water as granulation liquid.

f) Water-dispersible granules are also obtained by homogenizing and
precomminuting, in a colloid mill,
25 parts by weight of a compound of the formula (I) according to the invention,
5 parts by weight of sodium 2,2′-dinaphthylmethane-6,6′-disulfonate,
2 parts by weight of sodium oleoylmethyltauride,
1 part by weight of polyvinyl alcohol,
17 parts by weight of calcium carbonate and
50 parts by weight of water,
subsequently grinding the mixture in a bead mill, and atomizing and drying the resulting suspension in a spray tower by means of a single-substance nozzle.

### C. BIOLOGICAL EXAMPLES

### 1, Post-Emergence Herbicidal Action

Seeds or rhizome pieces of monocotyledonous and dicotyledonous weeds were placed in sandy loam in plastic pots (Jiffy pots), covered with soil and cultivated in a greenhouse under good and defined growth conditions, i.e. at a day/night rhythm of 22° C./14° C. At the 2-4 leaf stage, the plants were then treated with an aqueous test solution of various compounds according to the invention at the stated dosage. After four weeks, the tests were scored according to the scheme below:

0=no damage; 100=complete control

The control used was the Na⁺ salt of 3-(5,6-dihydro-1,4,2-dioxazin-3-yl)-N-[(4,6-dimethoxypyrimidin-2-yl)carbamoyl]pyridine-2-sulfonamide which is already known from U.S. Pat. No. 5,476,936.

| Compound | g of a.i./ha | ELEIN | AMBEL |
|---|---|---|---|
| control | 5 | 75 | 20 |
| I-1 | 5 | 85 | 80 |
| I-2 | 5 | 80 | 80 |
| I-5 | 5 | 80 | 75 |
| I-14 | 5 | 80 | 90 |
| I-35 | 5 | 80 | 85 |
| I-38 | 5 | 85 | 80 |
| I-39 | 5 | 93 | 80 |
| I-40 | 5 | 93 | 80 |

ELEIN = Eleusine indica
AMBEL = Ambrosia elatior
control = Na⁺ salt of 3-(5,6-dihydro-1,4,2-dioxazin-3-yl)-N-[(4,6-dimethoxypyrimidin-2-yl)carbamoyl]pyridine-2-sulfonamide
I-1, I-2, I-5, I-14, I-35, I-38, I-39, I-40 = comp ounds according to the invention according to Table 1

The sodium salt of 3-(5,6-dihydro-1,4,2-dioxazin-3-yl)-N-[(4,6-dimethoxypyrimidin-2-yl)carbamoyl]pyridine-2-sulfonamide already has a good controlling action at least as far as the monocotyledonous weed (Eleusine indica) is concerned; however, this is exceeded significantly when using the salts claimed by the invention of 3-(5,6-dihydro-1,4,2-dioxazin-3-yl)-N-[(4,6-dimethoxypyrimidin-2-yl)carbamoyl]pyridine-2-sulfonamide, i.e. the compounds I-1, I-2, I-5, I-14, I-35, I-38, I-39 and I-40 used here in an exemplary manner. The difference between the sodium salt, known from the literature, and the salts according to the invention is particularly pronounced when assessing the effectiveness against the dicotyledonous weed Ambrosia elatior, which is controlled relatively weakly by the sodium salt of 3-(5,6-dihydro-1,4,2-dioxazin-3-yl)-N-[(4,6-dimethoxypyrimidin-2-yl)carbamoyl]pyridine-2-sulfonamide, whereas the salts according to the invention of 3-(5,6-dihydro-1,4,2-dioxazin-3-yl)-N-[(4,6-dimethoxypyrimidin-2-yl)carbamoyl]pyridine-2-sulfonamide show at least a control of 75% (1-5) and in all other cases of at least 80%.

### 1. Pre-Emergence Herbicidal Action

Seeds or rhizome pieces of mono- and dicotyledonous weeds were placed in sandy loam in cardboard pots and covered with soil. The compounds according to the invention, formulated in the form of wettable powders or emulsion concentrates, were then applied to the surface of the covering soil in the form of aqueous suspensions or emulsions at an application rate of 100 to 800 l of water/ha (converted), at various dosages.

After the treatment, the pots were placed in a greenhouse and kept under good growth conditions for the weeds. The visual scoring of the damage to the plants or the emergence damage was carried out after the emergence of the test plants after a test period of 3 to 4 weeks, by comparison with untreated controls. As shown by the results, the compounds according to the invention have good herbicidal pre-emergence activity against a broad spectrum of weed grasses and broad-leaved weeds.

For example, the compounds Nos. I-1, I-2, I-5, I-14, I-35, I-38, I-39, I-40 from Table 1 have very good herbicidal activity against harmful plants such as Matricaria inodora, Papaver rhoeas, Stellaria media and Viola tricolor when applied by the pre-emergence method at an application rate of 0.08 kg or less of active substance per hectare.

3. Compatibility with Crop Plants

In further tests in the greenhouse, seeds of a relatively large number of crop plants and weeds were placed in sandy loam and covered with soil. Some of the pots were immediately treated as described in section 1, the others were placed in a greenhouse until the plants had developed two to three true leaves and were then sprayed with various dosages of the compounds according to the invention as described in section 2. Four to five weeks after the application and residence in the greenhouse, it was found by visual scoring that even high active compound dosages of the salts according to the invention, applied by the pre- and post-emergence method, did not damage leguminous plants. In these crops, the salts according to the invention display high selectivity, and they are therefore suitable for controlling unwanted vegetation in leguminous plants.

1. An agrochemically active salt of 3-(5,6-dihydro-1,4,2-dioxazin-3-yl)-N-[(4,6-dimethoxypyrimidin-2-yl)carbamoyyl]pyridine-2-sulfonamide of formula (I) 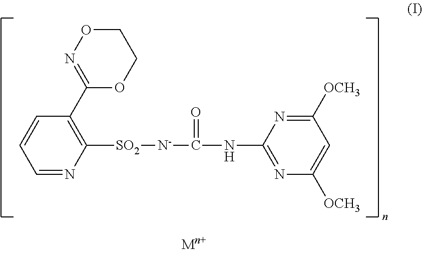
where the cation M
(a) is a lithium or potassium ion, or
(b) is an alkaline earth metal ion, or
(c) is a transition metal ion, or
(d) is an ammonium ion in which optionally one, two, three or all four hydrogen atoms are substituted by identical or different radicals from the group consisting of (C₁-C₄)-alkyl, hydroxy-(C₁-C₄)-alkyl, acylated hydroxy-(C₁-C₄)-alkyl, (C₃-C₆)-cycloalkyl, (C₁-C₄)-alkoxy-(C₁-C₄)-alkyl, hydroxy-(C₁-C₄)-alkoxy-(C₁-C₄)-alkyl, (C₁-C₆)-mercaptoalkyl, phenyl and benzyl, where the radicals mentioned above are optionally substituted by one or more identical or different radicals from the group consisting of halogen, nitro, cyano, azido, (C₁-C₆)-alkyl, (C₁-C₆)-haloalkyl, (C₃-C₆)-cycloalkyl, (C₁-C₆)-alkoxy, (C₁-C₆)-haloalkoxy and phenyl, and where in each case two substituents at the nitrogen atom together optionally form an unsubstituted or substituted ring, or
(e) is a phosphonium ion, or
(f) is a sulfonium ion, or
(g) is an oxonium ion, or
(h) is a saturated or unsaturated/aromatic nitrogenous heterocyclic ionic compound which has 1-10 carbon atoms in the ring system and is optionally mono- or polycondensed and/or mono- or polysubstituted by (C₁-C₄)-alkyl
and n corresponds to a number 1, 2 or 3.

2. The compound as claimed in claim 1 wherein
the cation M
(a) is a lithium or potassium ion, or
(b) is a calcium or magnesium ion, or
(c) is a manganese, copper, zinc or iron ion, or
(d) is an ammonium ion in which optionally one, two, three or all four hydrogen atoms are substituted by identical or different radicals from the group consisting of (C₁-C₄)-alkyl, hydroxy-(C₁-C₄)-alkyl, acylated hydroxy-(C₁-C₄)-alkyl, (C₃-C₄)-cycloalkyl, (C₁-C₂)-alkoxy-(C₁-C₂)-alkyl, hydroxy-(C₁-C₂)-alkoxy-(C₁-C₂)-alkyl, (C₁-C₂)-mercaptoalkyl, phenyl and benzyl, where the radicals mentioned above are optionally substituted by one or more identical or different radicals from the group consisting of halogen, nitro, cyano, azido, (C₁-C₂)-alkyl, haloalkyl, (C₃-C₄)-cycloalkyl, (C₁-C₂)-alkoxy, (C₁-C₂)-haloalkoxy and phenyl, and where in each case two substituents at the nitrogen atom together optionally form an unsubstituted or substituted ring, or
(e) is a tetra-((C₁-C₄)-alkyl)phosphonium ion or tetraphenylphosphonium ion, where the (C₁-C₄)-alkyl radicals and the phenyl radicals are optionally mono- or polysubstituted by identical or different radicals from the group consisting of halogen, (C₁-C₂)-alkyl, (C₁-C₂)-haloalkyl, (C₃-C₄)-cycloalkyl, (C₁-C₂)-alkoxy and (Cᵢ-C₂)-haloalkoxy, or
(f) is a tri-((C₁-C₄)-alkyl)sulfonium or triphenylsulfonium ion, where the (C₁-C₄)-alkyl radicals and the phenyl radicals are optionally mono- or polysubstituted by identical or different radicals from the group consisting of halogen, (C₁-C₂)-alkyl, (C₁-C₂)-haloalkyl, (C₃-C₄)-cycloalkyl, (C₁-C₂)-alkoxy and (C₁-C₂)-haloalkoxy, or
(g) is a tri-((C₁-C₄)-alkyl)oxonium ion, where the (C₁-C₄)-alkyl radicals are optionally mono- or polysubstituted by identical or different radicals from the group consisting of halogen, (C₁-C₂)-alkyl, (C₁-C₂)-haloalkyl, (C₃-C₄)-cycloalkyl, (C₁-C₂)-alkoxy and (C₁-C₂)-haloalkoxy, or
(h) is a cation from the group of the following heterocyclic compounds, pyridine, quinoline, 2-methylpyridine, 3-methylpyridine, 4-methylpyridine, 2,4-dimethylpyridine, 2,5-dimethylpyridine, 2,6-dimethylpyridine, 5-ethyl-2-methylpyridine, piperidine, pyrrolidine, morpholine, thiomorpholine, pyrrole, imidazole, 1,5-diazabicyclo[4.3.0]non-5-ene (DBN), 1,8-diazabicyclo[5.4.0]undec-7-ene (DBU)
and n corresponds to a number 1, 2 or 3.

3. The compound as claimed in claim 1 wherein
the cation M
is a potassium ion, a lithium ion, a magnesium ion, a calcium ion, an NH₄⁺ ion, a (2-hydroxyeth-1 -yl)ammonium ion, a bis-N,N-(2-hydroxyeth-1-yl)ammonium ion, a tris-N,N,N-(2-hydroxyeth-1-yl)ammonium ion, a methylammonium ion, a dimethylammonium ion, a trimethylammonium ion, a tetramethylammonium ion, an ethylammonium ion, a diethylammonium ion, a triethylammonium ion, a tetraethylammonium ion, an isopropylammonium ion, a diisopropylammonium ion, a tetrapropylammonium ion, a tetrabutylammonium ion, a 2-(2-hydroxyeth-1-oxy)eth-1-ylammonium ion, a di(2-hydroxyeth-1-yl)ammonium ion, a trimethylbenzylammonium ion, a tri-((C₁-C₄)-alkyl)sulfonium ion or a tri-((C₁-C₄)-alkyl)oxonium ion, a benzylammonium ion, a 1-phenylethylammonium ion, a 2-phenylethylammonium ion, a diisopropylethylammonium ion, a pyridinium ion, a piperidinium ion, an imidazolium ion, a morpholinium ion, a 1,8-diazabicyclo[5.4.0]undec-7-enium ion, a diethanolamine ion, an ethanolamine ion, a triethanolamine ion, a cholinium ion, a chlorocholinium ion or an acetylcholinium ion and n corresponds to a number 1 or 2.

4. The compound as claimed in claim 1 wherein
the cation M is a potassium ion, a magnesium ion, a calcium ion or an NH₄⁺ ion and n corresponds to a number 1 or 2.

5. The compound as claimed in claim 1 wherein
the cation M is a potassium ion and n is 1.

6. A process for preparing a compound as claimed in claim 1 comprising reacting a corresponding free acid with a base.

7. A method for controlling unwanted vegetation, which comprises allowing at least one compound as claimed in claim 1 to act upon an unwanted plant and/or a habitat thereof.

8. A compound as claimed in claim 1 that is suitable for controlling unwanted plants.

9. A herbicidal composition, which comprises a compound as claimed in claim 1 and at least one extender and/or surfactant.

10. A method for controlling unwanted vegetation, which comprises allowing at least one compound as claimed in claim 2 to act upon an unwanted plant and/or a habitat thereof.

11. A method for controlling unwanted vegetation, which comprises allowing at least one compound as claimed in claim 3 to act upon an unwanted plant and/or a habitat thereof.

12. A method for controlling unwanted vegetation, which comprises allowing at least one compound as claimed in claim 4 to act upon an unwanted plant and/or a habitat thereof.

13. A method for controlling unwanted vegetation, which comprises allowing at least one compound as claimed in claim 5 to act upon an unwanted plant and/or a habitat thereof.

14. A herbicidal composition, which comprises a compound as claimed in claim 2 and at least one extender and/or surfactant.

15. A herbicidal composition, which comprises a compound as claimed in claim 3 and at least one extender and/or surfactant.

16. A herbicidal composition, which comprises a compound as claimed in claim 4 and at least one extender and/or surfactant.

17. A herbicidal composition, which comprises a compound as claimed in claim 5 and at least one extender and/or surfactant.
